(19) United States
(12) Patent Application Publication
(10) Pub. No.: US 2025/0092007 A1
(43) Pub. Date: Mar. 20, 2025

(54) Arylamine Compound and Use thereof, Electroluminescent Device and Display Apparatus

(71) Applicant: BOE Technology Group Co., Ltd., Beijing (CN)

(72) Inventors: Yuqian SUN, Beijing (CN); Lixia QIU, Beijing (CN); Yang LIU, Beijing (CN)

(21) Appl. No.: 18/291,588

(22) PCT Filed: Sep. 14, 2022
(86) PCT No.: PCT/CN2022/118770

§ 371 Date: Jan. 24, 2024

(51) Int. Cl. C07D 311/96; C07C 211/61; C07D 217/18; C07D 263/64; C07D 405/12; C07D 413/14; H10K 50/15; H10K 50/17; H10K 50/18

(52) U.S. Cl. CPC C07D 311/96; C07C 211/61; C07D 217/18; C07D 263/64; C07D 405/12; C07D 413/14; C07C 2601/16; C07C 2603/94; H10K 50/15; H10K 50/17; H10K 50/181

(57) ABSTRACT

An arylamine compound and use thereof, an electroluminescent device and a display apparatus are provided, the arylamine compound has a general structural formula of Formula I; wherein each group and substituent have the same meaning as that in the description.

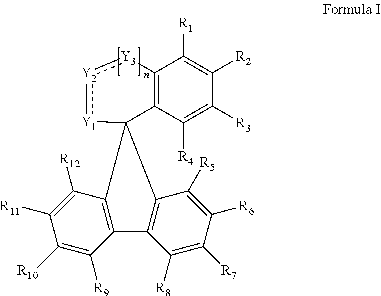

### CROSS-REFERENCE TO RELATED APPLICATION

The present application is a U.S. National Phase Entry of International Application No. PCT/CN2022/118770, having an international filing date of Sep. 14, 2022, and entitled “Arylamine Compound and Use thereof, Electroluminescent Device and Display Apparatus”, the content of which is hereby incorporated herein by reference in its entirety.

### TECHNICAL FIELD

Embodiments of the present disclosure relate to, but are not limited to, the field of display technology, in particular to an arylamine compound and use thereof, an electroluminescent device and a display apparatus.

### BACKGROUND

Organic Light Emitting Diode (OLED) is an active light emitting display device, which has the characteristics such as self-luminescence, high luminous brightness and efficiency, high resolution, wide color gamut and viewing angle, fast response speed, low energy consumption and flexibility, and has become a mainstream display product in the market at present.

Generally, organic luminescence refers to the phenomenon that electric energy is converted into light energy by using organic substances. OLED devices utilizing organic luminescence generally have an anode, a cathode and a functional layer containing organic substance disposed between the anode and the cathode. In order to improve the efficiency and stability of OLED, the functional layer containing organic substance generally adopts multi-layer structure composed of various substances. For example, it can be formed by Hole Injection Layer (HIL), Hole Transport Layer (HTL), Emitting Layer (EML), Electron Transport Layer (ETL) and Electron Injection Layer (EIL), etc., and Hole Block Layer (HBL) and Electron Block Layer (EBL) can also be added.

At present, many organic materials suitable for OLED functional layers have been developed, such as polycyclic compounds containing heteroatom(s). However, the properties of polycyclic compounds are greatly influenced by their chemical structures. For example, they may have different characteristics depending on the number and fusion position of rings, the species and arrangements of heteroatoms, band gaps (HOMO, LUMO), chemical properties and physical properties. Therefore, the research on developing materials suitable for OLED functional layers based on the diversity of organic substance has been continuously carried out.

### SUMMARY

The following is a summary of subject matter described herein in detail. The summary is not intended to limit the protection scope of the present application.

An embodiment of the present disclosure provides an arylamine compound, having a general structural formula of:

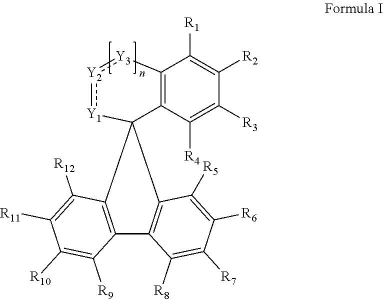

wherein, n is 0, 1 or 2;
at least one of R₁ to R₁₂ is -L-N(Ar₁)-Ar₂, and the R₁ to R₁₂ that are not -L-N(Ar₁)-Ar₂ are each independently selected from any one of hydrogen, deuterium, an alkyl group with 1 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group;
Ar₁ and Ar₂ are each independently selected from any one of an alkyl group with 4 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar₁ and Ar₂ and the N connected with Ar₁ and Ar₂ can form a ring;
L is selected from any one of a single bond, a substituted or unsubstituted arylene group with 6 to 39 carbon atoms, and a substituted or unsubstituted heteroarylene group with 5 to 60 carbon atoms; wherein, the substituted arylene group with 6 to 39 carbon atoms and the substituted heteroarylene group with 5 to 60 carbon atoms mean being substituted by an aryl group with 6 to 39 carbon atoms and a heteroaryl group with 5 to 60 carbon atoms;
Y₁, Y₂, and Y₃ satisfy one of the following conditions:
1) the dotted line between Y₁ and Y₂ exists, and a double bond is formed between Y₁ and Y₂; and the dotted line between Y₂ and Y₃ does not exist;
Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—;
2) the dotted line between Y₁ and Y₂ does not exist; and the dotted line between Y₂ and Y₃ exists, and a double bond is formed between Y₂ and Y₃;
Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; and
3) the dotted line between Y₁ and Y₂ does not exist, and the dotted line between Y₂ and Y₃ does not exist;
Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—;
R₁₃ to R₁₇ are each independently selected from any one of hydrogen, deuterium, an alkyl group with 1 to 39 carbon atoms, and an aryl group with 6 to 39 carbon atoms.

In embodiments of the present disclosure, Y₁, Y₂, and Y₃ may satisfy at least one of the following conditions:
1) at least one of Y₁, Y₂, and Y₃ is selected from —N(R₁₅)—, —O— and S—, and Y₁, Y₂, and Y₃ are not simultaneously selected from —O— and —S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

In embodiments of the present disclosure, Y₁, Y₂, and Y₃ may satisfy at least one of the following conditions:
1) one of Y₁, Y₂, and Y₃ is selected from any one of —N(R₁₃)—, —O— and S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

In embodiments of the present disclosure, the structural formula of the arylamine compound may be:

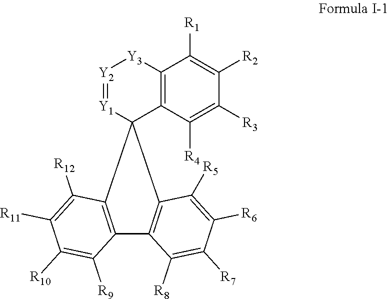

wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

In embodiments of the present disclosure, the structural formula of the arylamine compound may be:

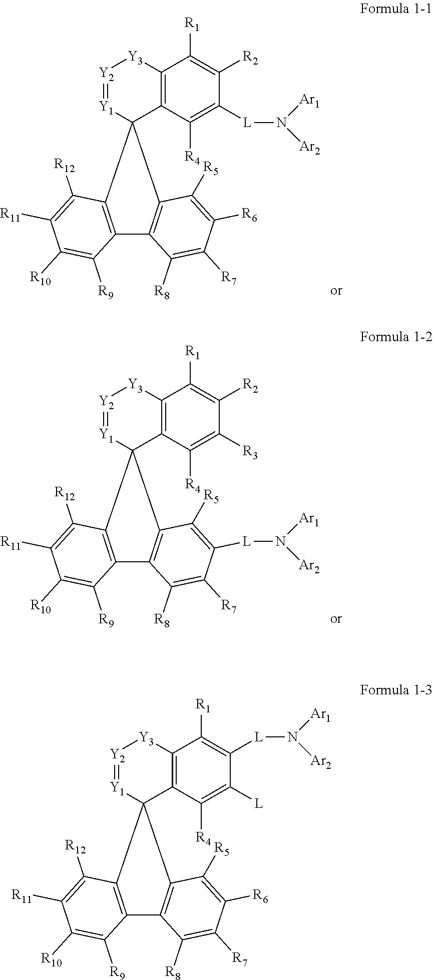

wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

In embodiments of the present disclosure, the structural formula of the arylamine compound may be:

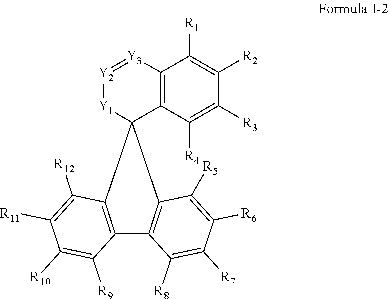

wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R₁ to R₁₂ and R₁₅ to R₁₇ are the same as those in Formula I.

In embodiments of the present disclosure, the structural formula of the arylamine compound may be:

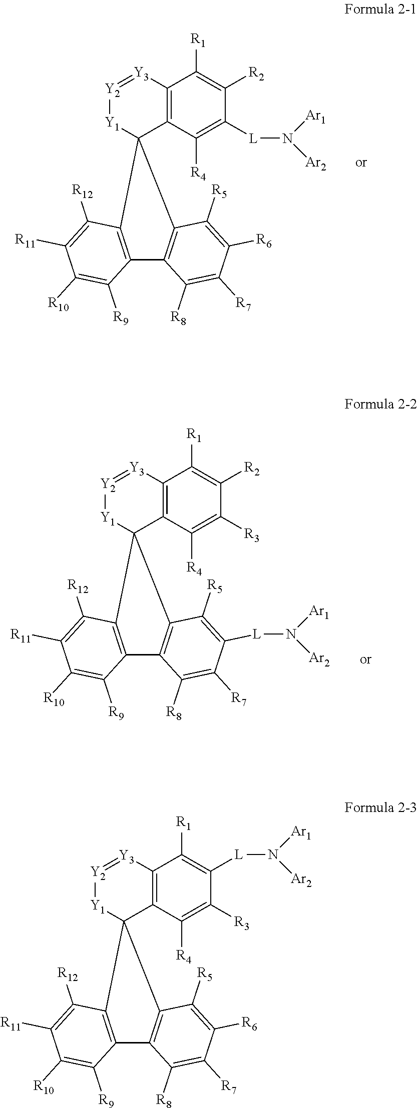

wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R₁ to R₁₂ and R₁₅ to R₁₇ are the same as those in Formula I.

In embodiments of the present disclosure, the structural formula of the arylamine compound may be:

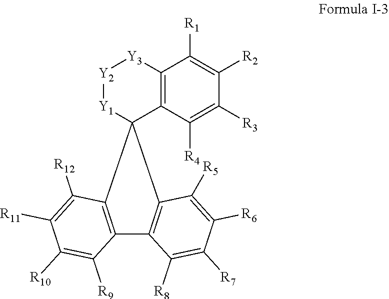

wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

In embodiments of the present disclosure, the structural formula of the arylamine compound may be:

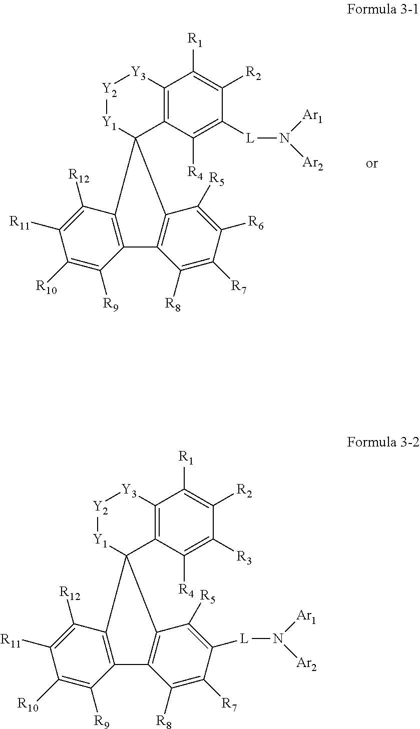

wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

In embodiments of the present disclosure, Ar₁ and Ar₂ may be each independently selected from any one of a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar₁ and Ar₂ and the N connected with Ar₁ and Ar₂ can form a ring.

In embodiments of the present disclosure, Ar₁ and Ar₂ may be each independently selected from any one of the following groups:

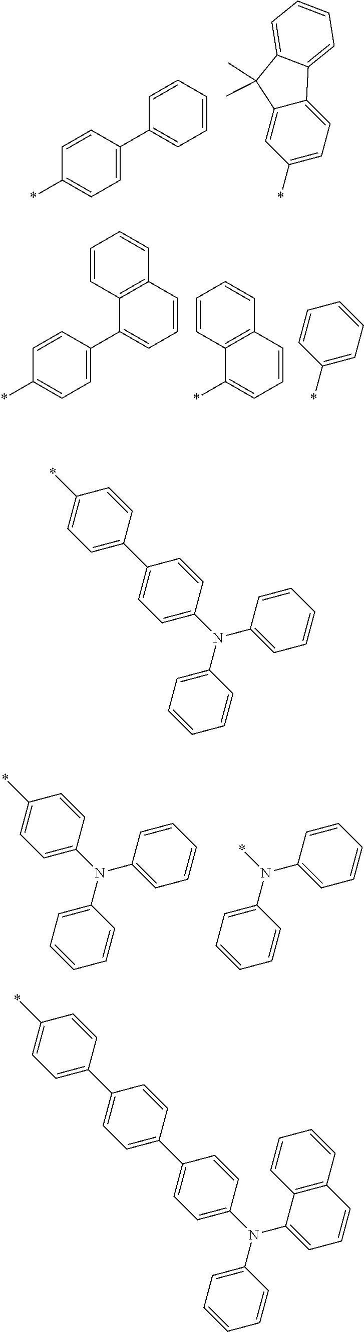

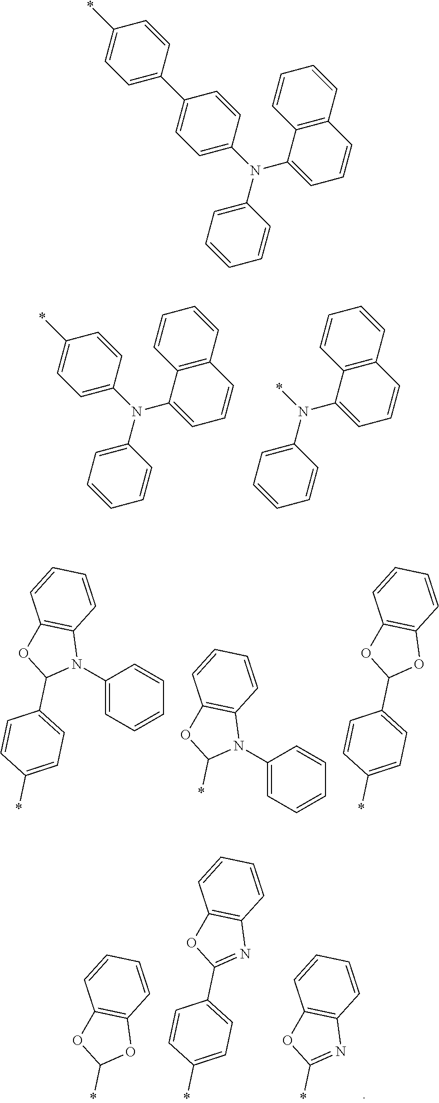

In embodiments of the present disclosure, R₁₃ to R₁₈ may be each independently selected from any one of hydrogen, deuterium, an alkyl group with 1 to 4 carbon atoms, and an aryl group with 6 to 20 carbon atoms.

In embodiments of the present disclosure, R₁ to R₁₂ that are not -L-N(Ar₁)-Ar₂ may be independently selected from any one of hydrogen and deuterium.

In embodiments of the present disclosure, L may be selected from any one of a single bond, a phenylene group, a benzene or phenyl substituted phenylene group, and a biphenyl substituted phenylene group.

In embodiments of the present disclosure, the arylamine compound may include any one of the following compounds:

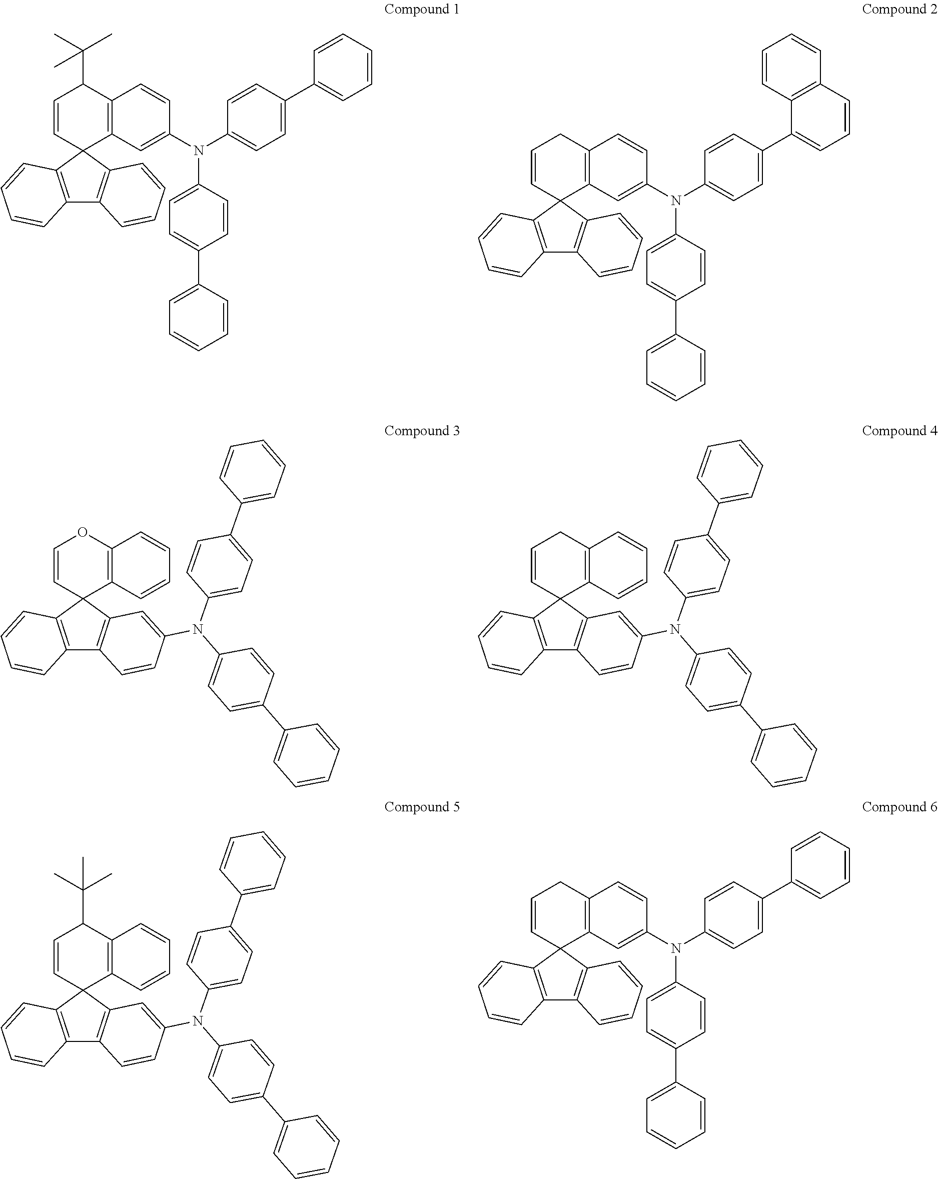

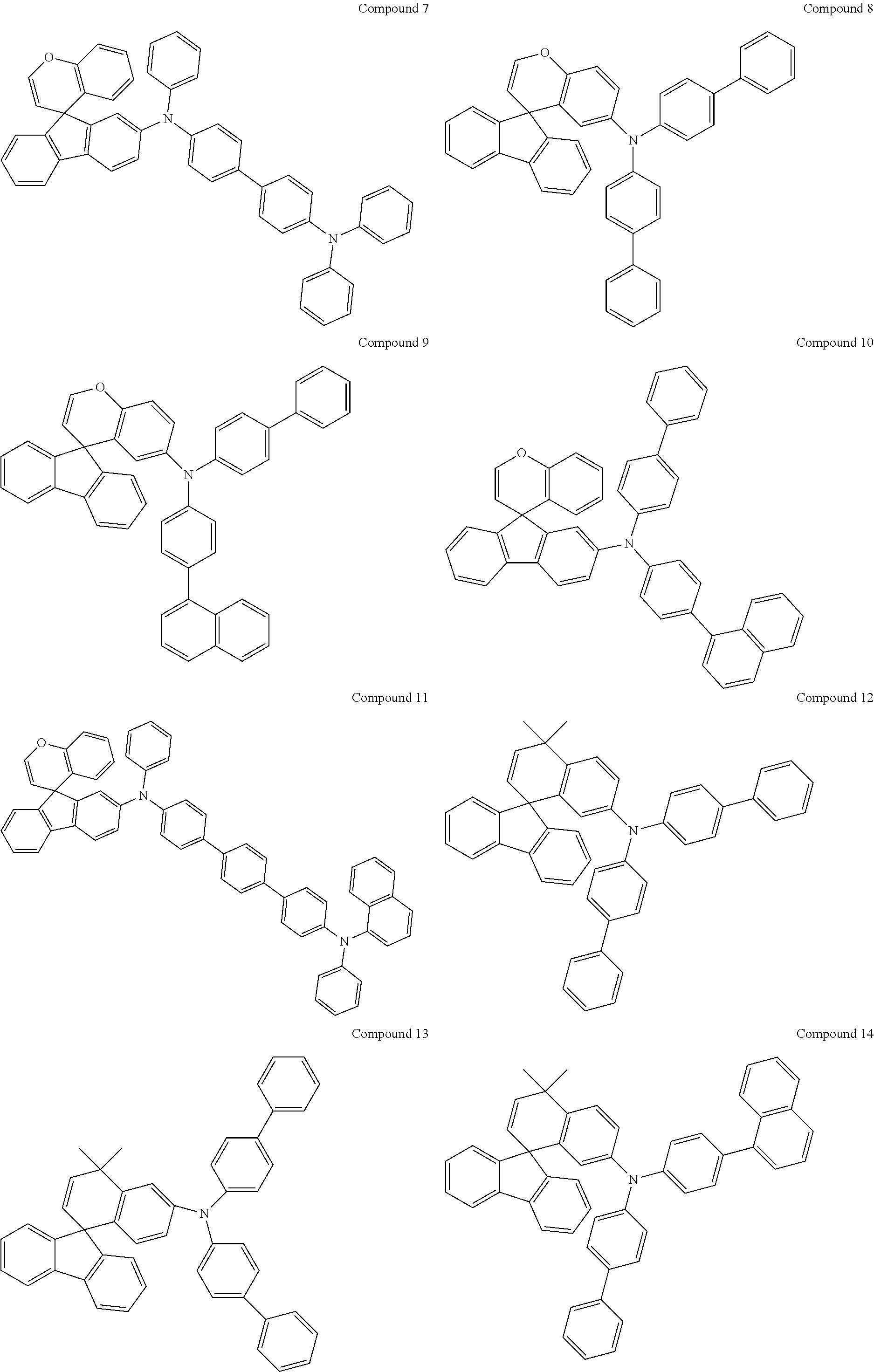

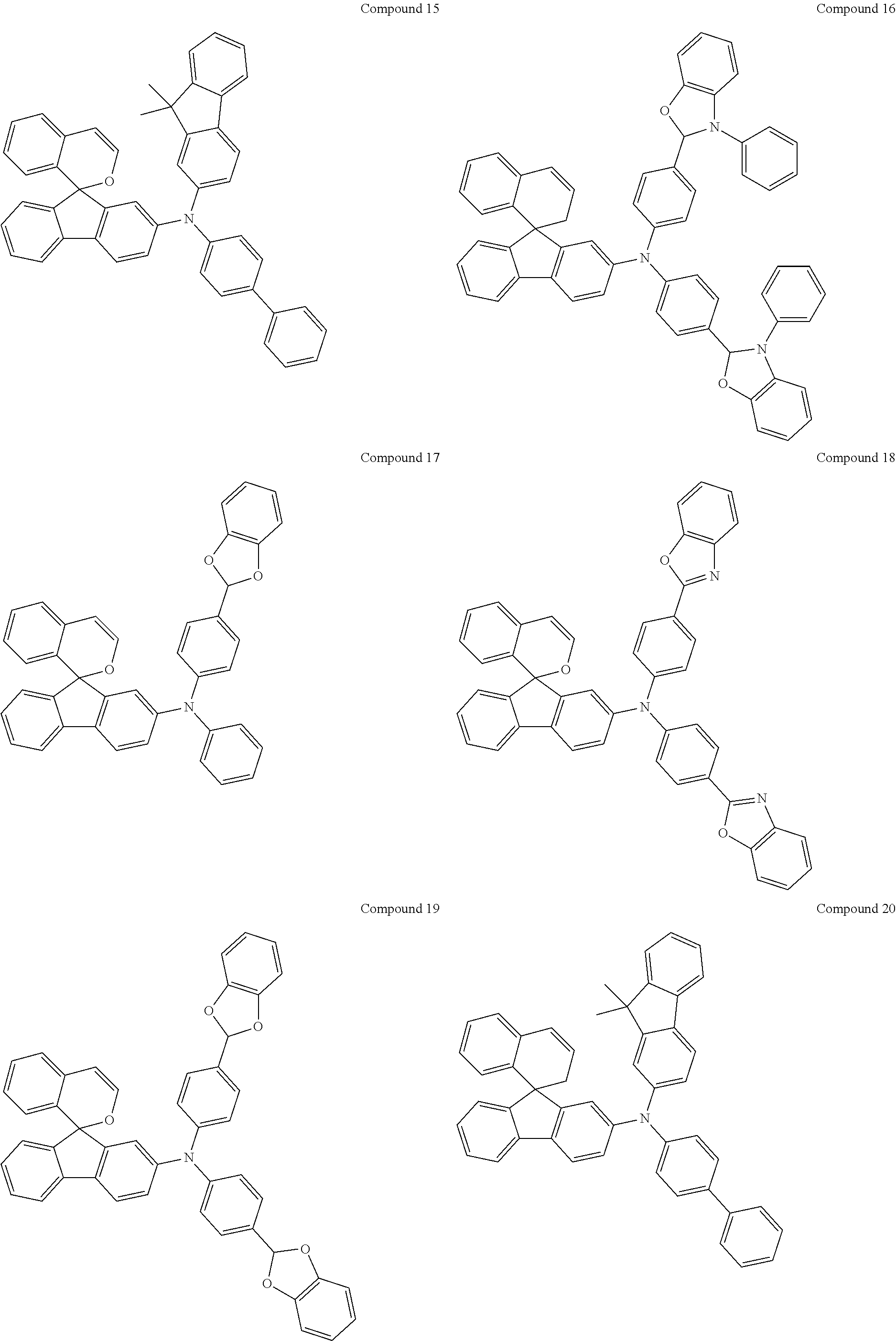

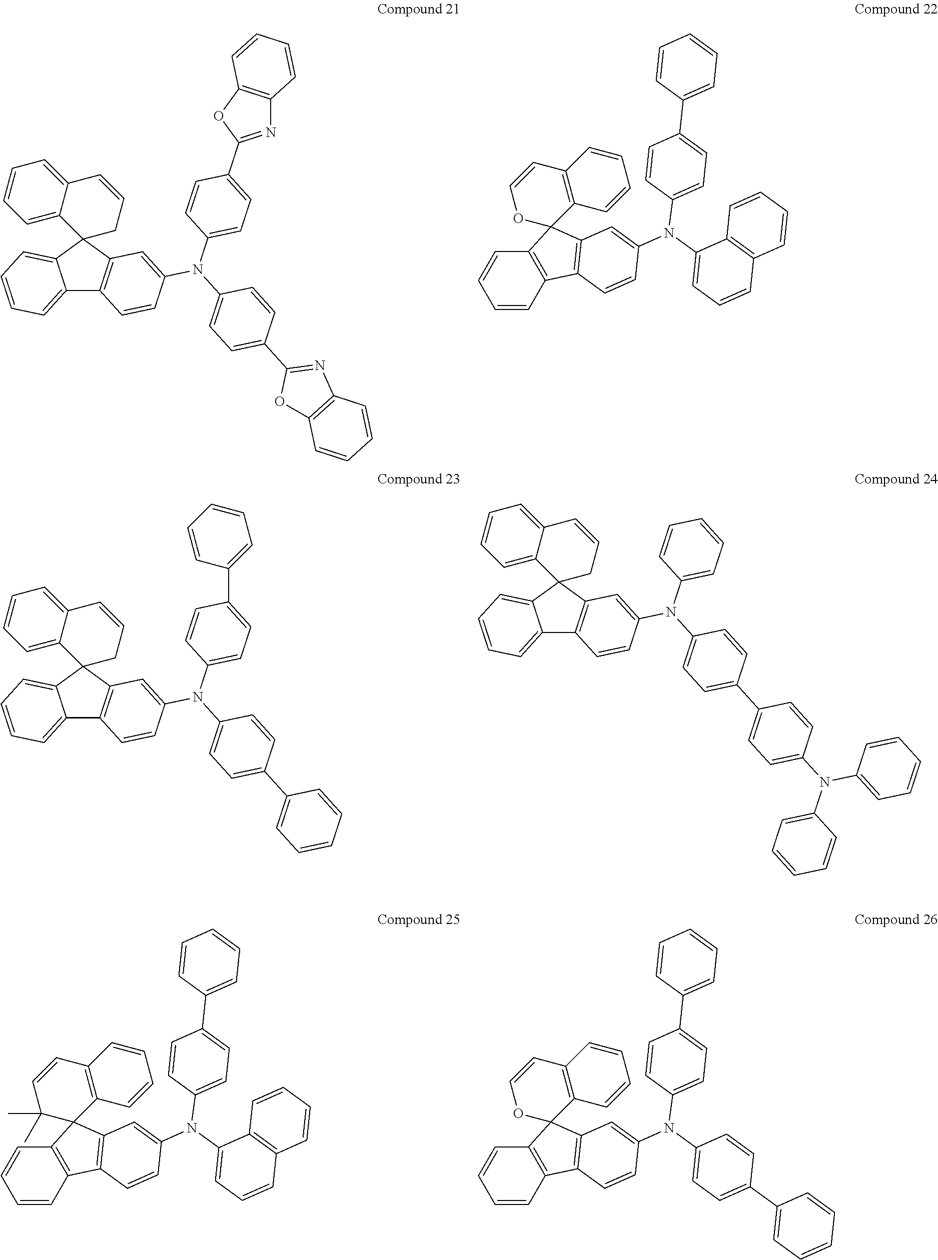

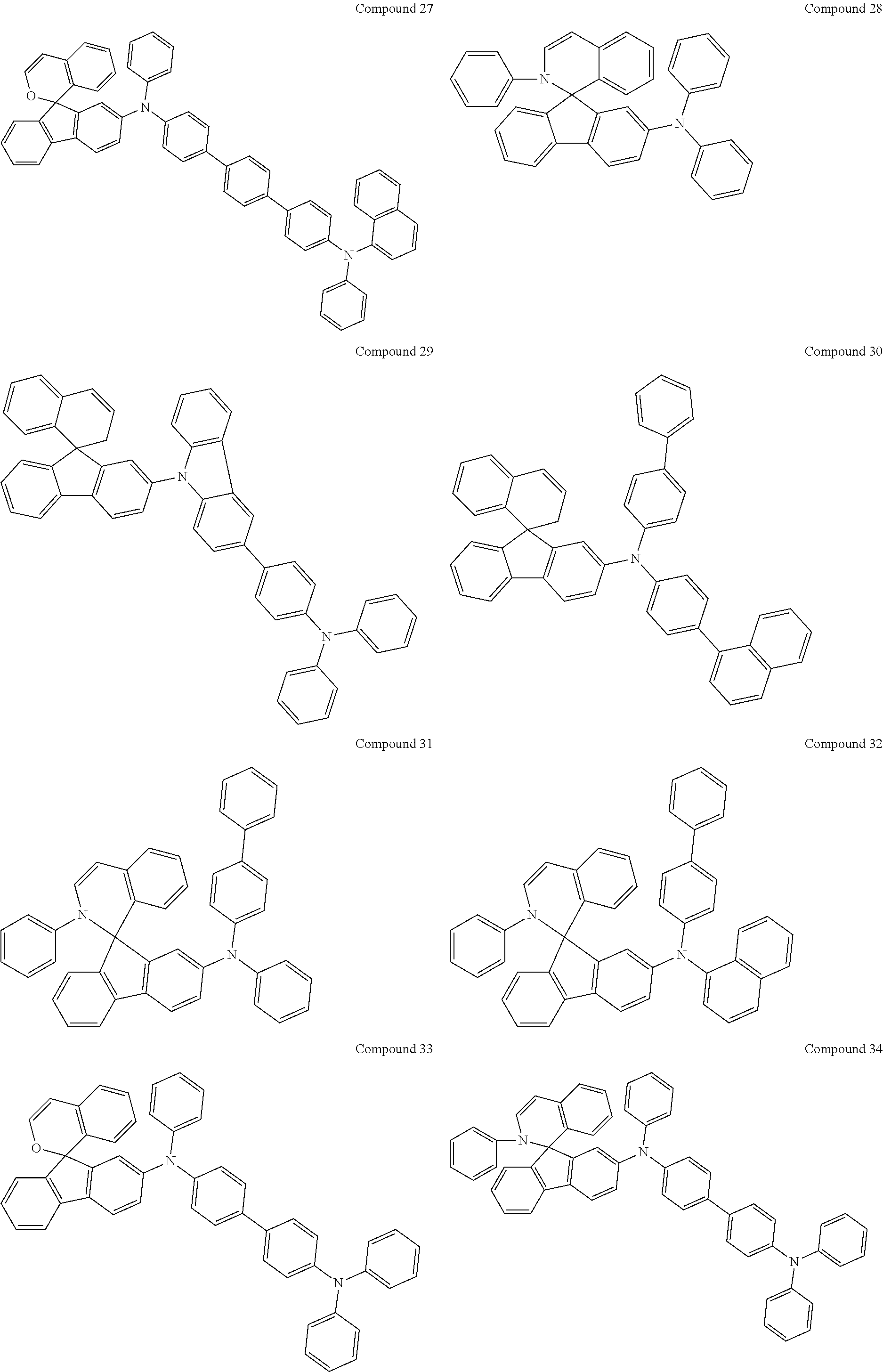

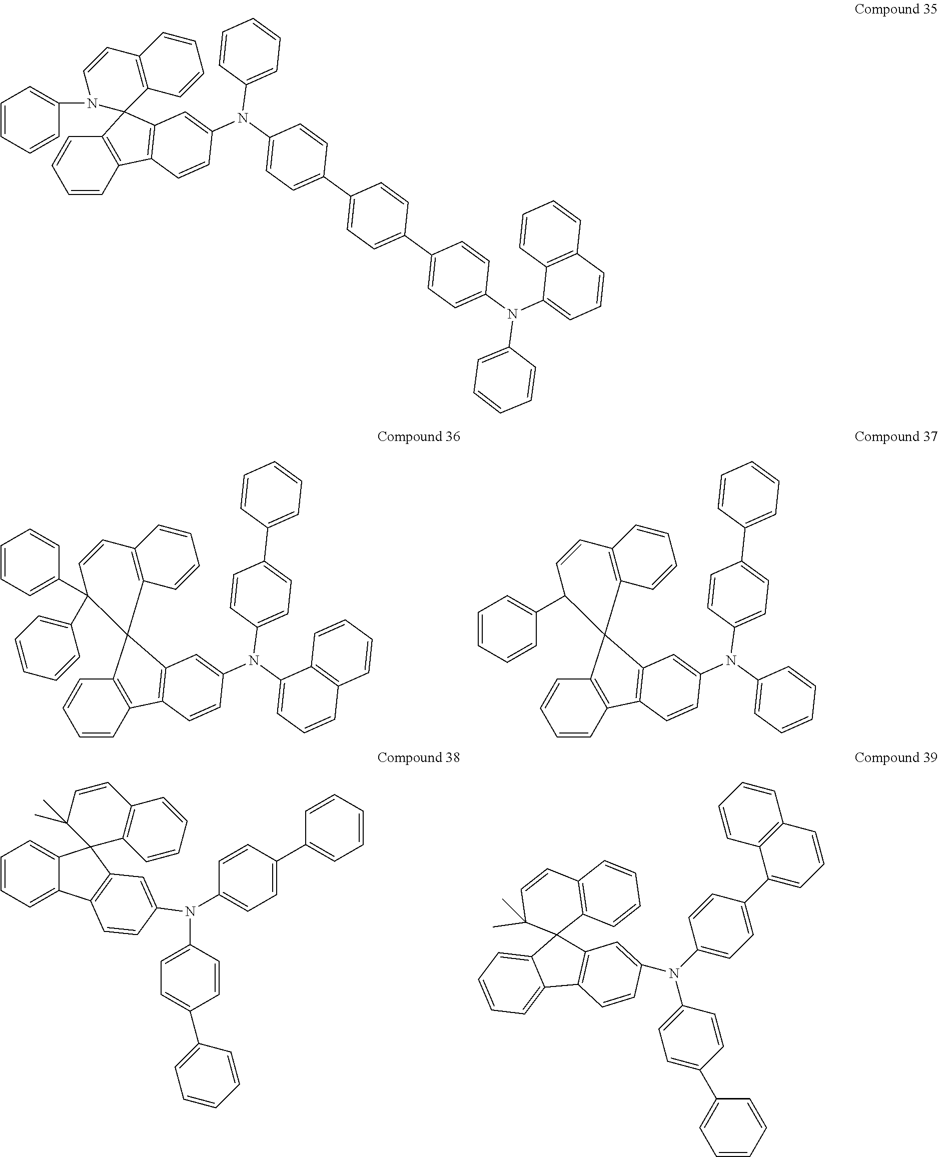

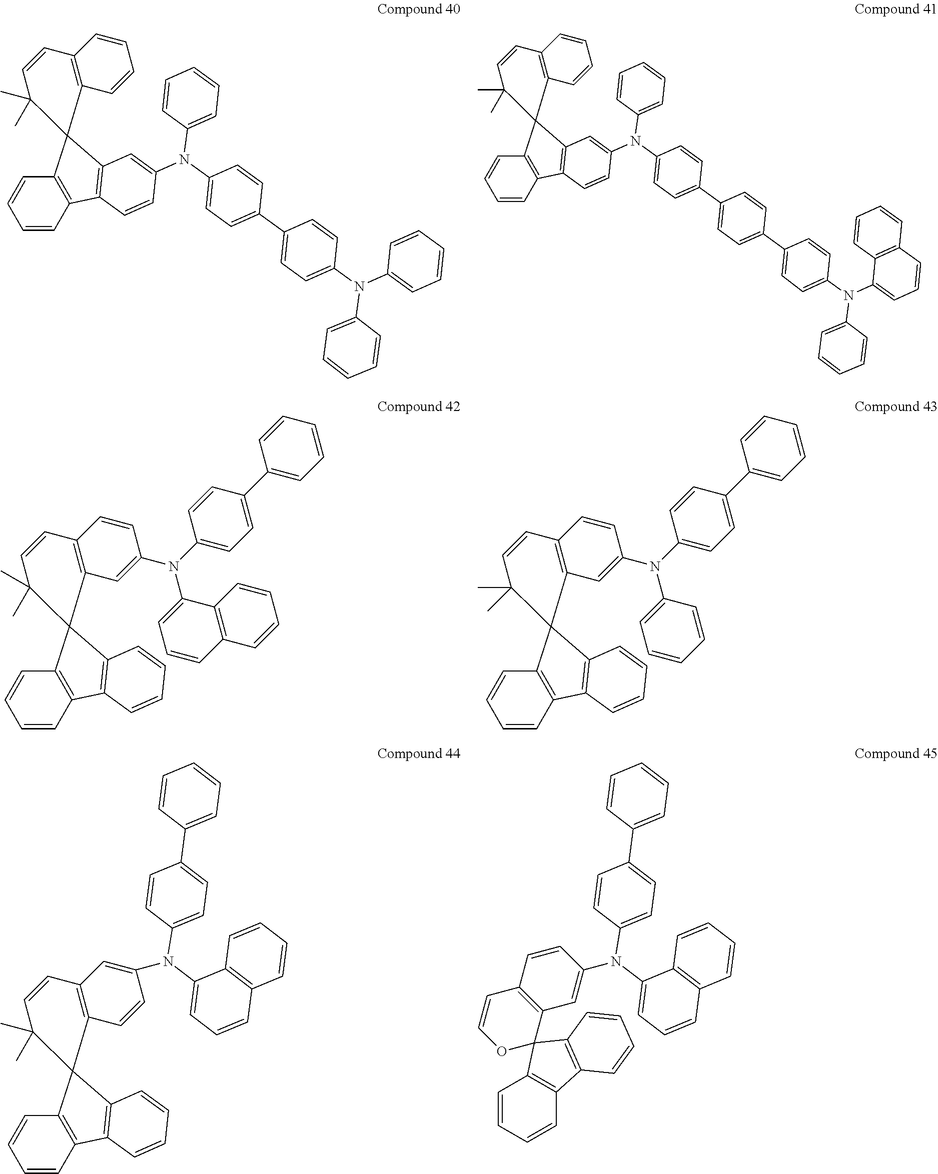

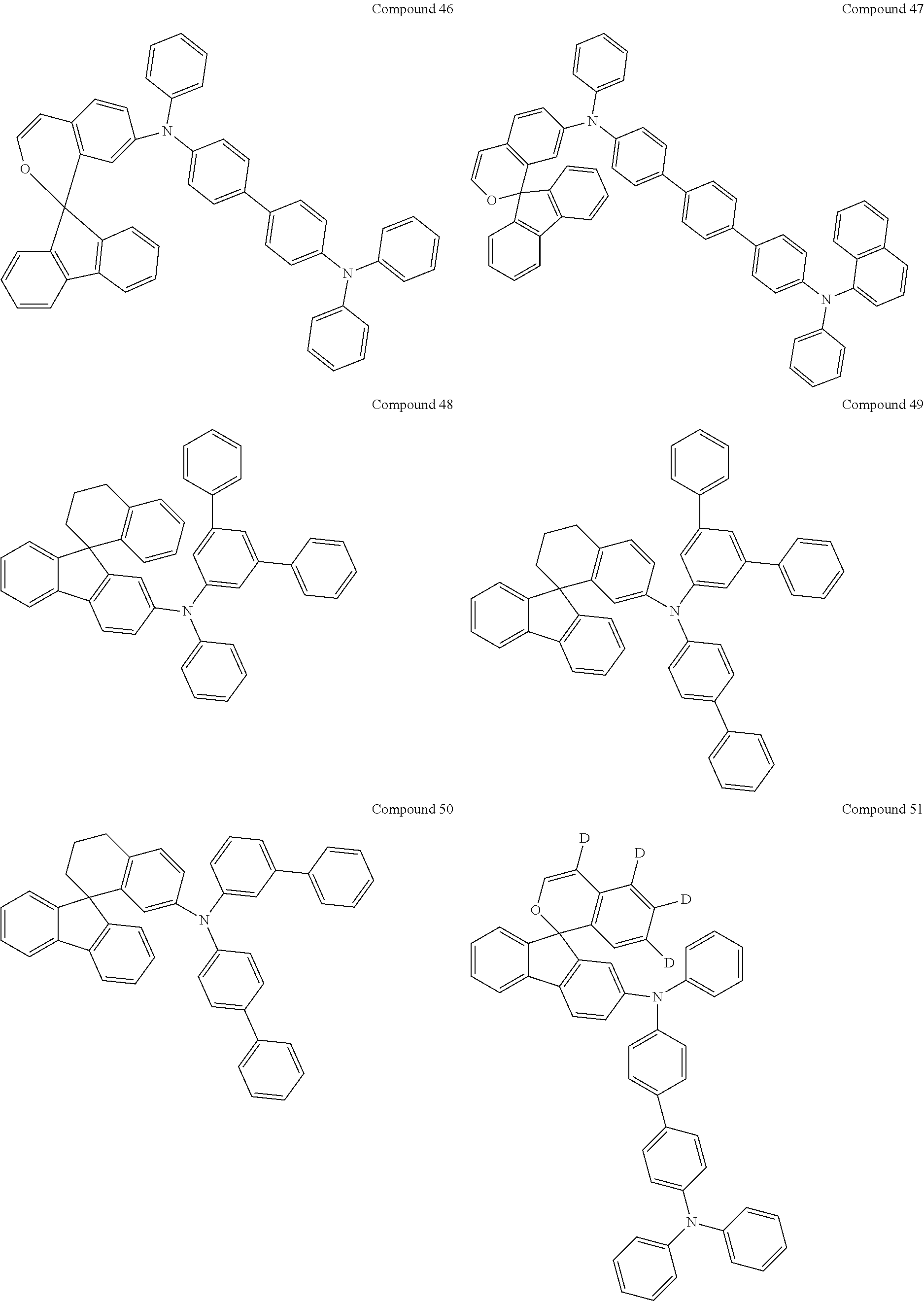

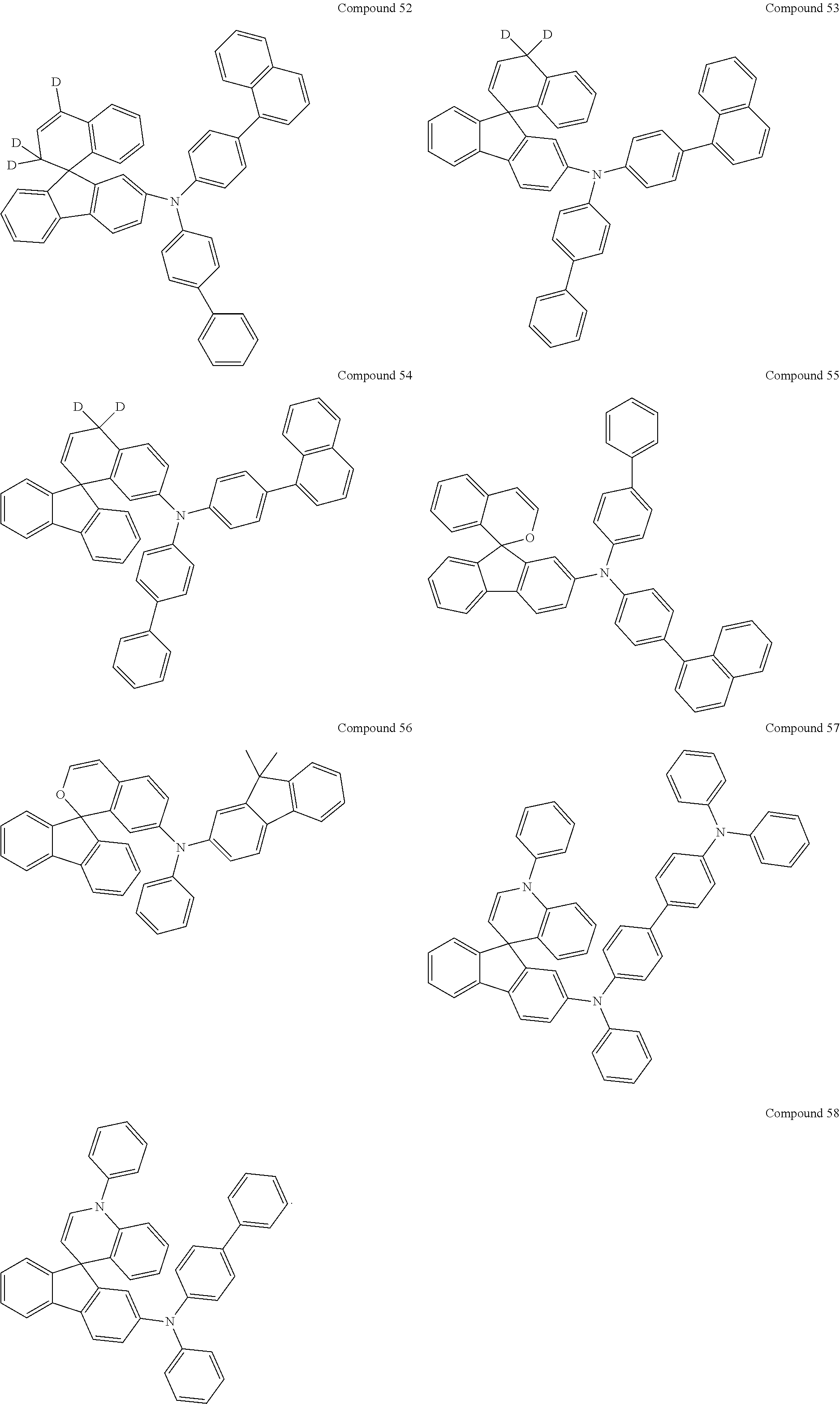

### Compound 57 Compound 58.

In embodiments of the present disclosure, the glass transition temperature of the arylamine compound may be 120° C. to 180° C.

In embodiments of the present disclosure, the glass transition temperature of the arylamine compound may be 125° C. to 140° C.

In embodiments of the present disclosure, the highest occupied molecular orbital energy level of the arylamine compound may be −4.6 eV to −5.7 eV.

In embodiments of the present disclosure, the highest occupied molecular orbital energy level of the arylamine compound may be −4.6 eV to −4.9 eV or −5.3 eV to −5.5 eV.

In embodiments of the present disclosure, the triplet energy level of the arylamine compound may be 1.8 eV to 2.6 eV.

In embodiments of the present disclosure, the triplet energy level of the arylamine compound may be 2.1 eV to 2.6 eV.

In embodiments of the present disclosure, the triplet energy level of the arylamine compound may be 2.4 eV to 2.6 eV.

An embodiment of the present disclosure further provides use of the arylamine compound as described above as a hole injection material.

An embodiment of the present disclosure further provides use of the arylamine compound as described above as a hole transport material.

An embodiment of the present disclosure further provides use of the arylamine compound as described above as an electron block material.

An embodiment of the present disclosure further provides an electroluminescent device, including the arylamine compound as described above.

In embodiments of the present disclosure, the electroluminescent device may include a hole injection layer, a hole transport layer and an electron block layer, wherein:
the hole injection layer and the hole transport layer each comprise the arylamine compound as described above; or,
the hole injection layer and the electron block layer each comprise the arylamine compound as described above; or,
the hole injection layer, the hole transport layer, and the electron block layer each comprise the arylamine compound as described above.

An embodiment of the present disclosure further provides a display apparatus, including the electroluminescent device described above.

After the drawings and the detailed descriptions are read and understood, the other aspects may be comprehended.

### BRIEF DESCRIPTION OF DRAWINGS

The accompany drawings are used to provide further understanding of the technical solution of the present disclosure, and form a part of the description. The accompany drawings and embodiments of the present disclosure are adopted to explain the technical solution of the present disclosure, and do not form limits to the technical solution of the present disclosure.

FIG. 1 is a schematic diagram of structure of an electroluminescent device according to an exemplary embodiment of the present disclosure;

FIG. 2 is curves of transverse current of the compound of Comparative Example 1 versus compound 2 of an embodiment of the present disclosure as a function of a doping ratio of a P-type dopant.

Meanings of reference signs in the accompanying drawings are as follows:
100—anode; 200—hole injection layer; 300—hole transport layer; 400—electron block layer; 500—emitting layer; 600—hole block layer; 700—electron transport layer; 800—electron injection layer; 900—cathode; and 1000—light extraction layer.

### DETAILED DESCRIPTION

Implementations herein may be implemented in multiple different forms. Those of ordinary skills in the art can readily appreciate a fact that the implementations and contents may be varied into various forms without departing from the spirit and scope of the present disclosure. Therefore, the present disclosure should not be explained as being limited to the contents recorded in the following implementations only. The embodiments and features in the embodiments of the present disclosure may be randomly combined with each other in case of no conflicts.

Scales of the drawings in the present disclosure may be used as a reference in actual processes, but are not limited thereto. For example, a width-length ratio of a channel, a thickness and spacing of each film layer, and a width and spacing of each signal line may be adjusted according to actual needs. A quantity of pixels in a display substrate and a quantity of sub-pixels in each pixel are not limited to numbers shown in the drawings. The drawings described in the present disclosure are schematic structural diagrams only, and one mode of the present disclosure is not limited to shapes, numerical values, or the like shown in the drawings.

In the specification, for convenience, wordings indicating orientation or positional relationships, such as “middle”, “upper”, “lower”, “front”, “back”, “vertical”, “horizontal”, “top”, “bottom”, “inside”, “outside”, and the like, are used for illustrating positional relationships between constituent elements with reference to the drawings, and are merely for facilitating the description of the specification and simplifying the description, rather than indicating or implying that a referred apparatus or element must have a particular orientation and be constructed and operated in the particular orientation. Therefore, they cannot be understood as limitations on the present disclosure. The positional relationships between the composition elements may be changed as appropriate according to the direction where each composition element is described. Therefore, appropriate replacements based on situations are allowed, not limited to the expressions in the specification.

In the specification, unless otherwise specified and defined explicitly, terms “set” and “connect” should be understood in a broad sense. For example, the term may be fixed connection, or detachable connection, or integral connection. The term may be mechanical connection or electric connection. The term may be direct connection, or indirect connection through an intermediate, or communication inside two elements. Those of ordinary skill in the art can understand specific meanings of the above terms in the present disclosure according to specific situations.

In the specification, a “film” and a “layer” are interchangeable. For example, an “emitting layer” may be replaced with a “light emitting film” sometimes.

The photoelectric functional material(s) used in OLED devices includes at least a hole injection material, a hole transport material, a luminescent material, an electron transport material, etc. Type and combination form of the materials have the characteristics of richness and diversity. In addition, for OLED devices with different structures, the photoelectric functional materials used have relatively strong selectivity, and performance of the same materials in devices with different structures may be completely different.

Therefore, according to current requirements of OLED devices for industrial application, requirements of OLED devices for photoelectric characteristics, and requirements of OLED devices for characteristics of different functional films, there is a need to select more suitable and high-performance OLED functional materials or material combinations, in order to achieve the comprehensive characteristics of high efficiency, long lifetime and low voltage of the devices. In terms of the actual demand of current OLED display lighting industry, the development of OLED materials is far from enough and lags behind the requirements of panel manufacturers, therefore, it is particularly important to develop organic functional materials of higher performance.

In addition, crosstalk occurs in the current mass-produced OLED devices. The reason for this phenomenon is that P-doped hole transport materials are often used as hole injection materials. After P-doping, the transverse current of conventional hole transport materials becomes hundreds of times larger, which leads to transverse migration of charges. However, the turn-on voltage of three colors of RGB gradually increases. When blue sub-pixels turn on, green sub-pixel and red sub-pixel turn on at the same time under the drive of transverse current, resulting in color crosstalk.

An embodiment of the present disclosure provides an arylamine compound, having a general structural formula of:

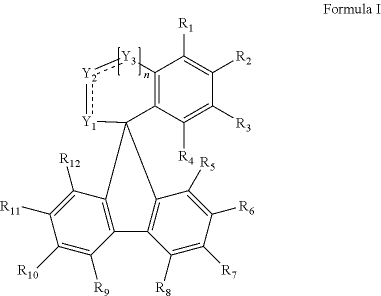

wherein, n is 0, 1 or 2;
at least one of R₁ to R₁₂ is -L-N(Ar₁)-Ar₂, and the R₁ to R₁₂ that are not -L-N(Ar₁)-Ar₂ are each independently selected from any one of hydrogen, deuterium, an alkyl group with 1 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group;
Ar₁ and Ar₂ are each independently selected from any one of an alkyl group with 4 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar and Ar₂ and the N connected with Ar₁ and Ar₂ can form a ring;
L is selected from any one of a single bond, a substituted or unsubstituted arylene group with 6 to 39 carbon atoms, and a substituted or unsubstituted heteroarylene group with 5 to 60 carbon atoms; wherein, the substituted arylene group with 6 to 39 carbon atoms and the substituted heteroarylene group with 5 to 60 carbon atoms mean being substituted by an aryl group with 6 to 39 carbon atoms and a heteroaryl group with 5 to 60 carbon atoms;
Y₁, Y₂, and Y₃ satisfy one of the following conditions:
1) the dotted line between Y₁ and Y₂ exists, and a double bond is formed between Y₁ and Y₂; and the dotted line between Y₂ and Y₃ does not exist;
Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—;
2) the dotted line between Y and Y₂ does not exist; and the dotted line between Y₂ and Y₃ exists, and a double bond is formed between Y₂ and Y₃;
Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; and
3) the dotted line between Y₁ and Y₂ does not exist, and the dotted line between Y₂ and Y₃ does not exist;
Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—;
R₁₃ to R₁₇ are each independently selected from any one of hydrogen, deuterium, an alkyl group with 1 to 39 carbon atoms, and an aryl group with 6 to 39 carbon atoms.

In embodiments of the present disclosure, the aryl group includes, but is not limited to, phenyl group, naphthyl group, anthryl group, acenaphthylenyl group, indenyl group, phenanthrenyl group, azulenyl group, pyrenyl group, fluorenyl group, perylene group, spirofluorenyl group, spirobifluorenyl group, chrysenyl group, benzophenanthrenyl group, benzoanthryl group, fluoranthenyl group, picenyl group, naphthacenyl group, and indenophenyl group.

The term “hetero” as used in heteroaryl group means that at least one carbon atom in an aromatic ring is substituted by a heteroatom selected from any one or more of a nitrogen atom (N), an oxygen atom (O), and a sulfur atom (S).

In embodiments of the present disclosure, the heteroaryl includes, but is not limited to, benzoxazolyl group, benzothiazolyl group, indolyl group, benzimidazolyl group, pyrrolyl group, pyridyl group, pyrimidinyl group, pyrazinyl group, pyridazinyl group, triazinyl group, tetrazinyl group, imidazolyl group, pyrazolyl group, carbazolyl group, thienyl group, thiazolyl group, benzocarbazolyl group, dibenzocarbazolyl group, indolo carbazolyl group, indeno carbazolyl group, quinolinyl group, isoquinolinyl group, phthalazinyl group, quinoxalinyl group, cinnolinyl group, quinazolinyl group, phthalazinyl group, benzoquinolinyl group, benzisoquinolinyl group, benzoquinazolinyl group, benzoquinoxalinyl group, acridyl group, phenanthrolinyl group, furyl group, pyranyl group, oxazinyl group, oxazolyl group, oxadiazolyl group, triazolyl group, dioxynyl group, benzofuryl group, dibenzofuryl group, thiopyranyl group, thiazinyl group, phenylthio group and N-substituted spirofluorenyl group.

The arylamine compound provided by embodiments of the present disclosure is a kind of aromatic hydrocarbon based molecular blocks with sp3 hybrid carbon atom as the center and with a cross configuration. The molecule has certain stereoscopic property, which can improve the thermal stability of the molecule, enabling the molecule to obtain higher glass transition temperature (Tg).

Furthermore, the charge in the arylamine compound provided by embodiments of the present disclosure is not easy to diffuse transversely after film formation, so that the lifetime, efficiency and/or working voltage can be improved at the same time. If a substituent(s) is/are introduced in the arylamine compound provided by embodiments of the present disclosure, the transverse current of the compound can be further reduced, which is about 10% of that of conventional materials. Heteroatom(s) can also be introduced, which can increase the injection of positive charges, so that the compound can be applied to a hole injection material, a hole transport material or an electron block material.

In addition, some applications of spirocyclic compounds in an electron injection layer, an electron transport layer, an electron block layer or a hole transport layer have currently been reported in literatures, for example, Chinese invention patent applications CN112358471A and CN113773208A, etc. However, the spirocyclic compounds reported in CN112358471A and CN113773208A require relatively higher evaporation temperature (Te) during deposition, so the mass-production process requires higher requirements, otherwise it will easily lead to material decomposition or defect state, resulting in low yield or insufficient lifetime, etc. However, the arylamine compound provided by embodiments of the present disclosure has low molecular weight and is beneficial to forming a melt-type evaporation material, so that the evaporation temperature (Te) required during deposition is relatively low, and the glass transition temperature (Tg) is relatively high, which has higher process feasibility and can improve the stability of mass-production evaporation.

In an exemplary embodiment of the present disclosure, Y₁, Y₂, and Y₃ may satisfy at least one of the following conditions:
1) at least one of Y₁, Y₂, and Y₃ is selected from —N(R₁₅)—, —O— and S—, and Y₁, Y₂, and Y₃ are not simultaneously selected from —O— and —S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

In an exemplary embodiment of the present disclosure, Y₁, Y₂, and Y₃ may satisfy at least one of the following conditions:
1) one of Y₁, Y₂, and Y₃ is selected from any one of —N(R₁₃)—, —O— and S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

In an exemplary embodiment of the present disclosure, the structural formula of the arylamine compound may be:

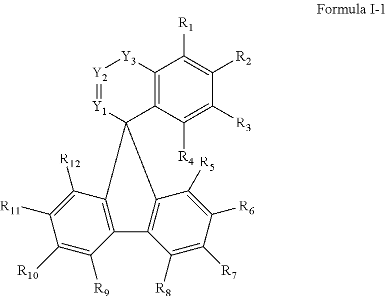

wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

In the compound represented by Formula I-1, a double bond is formed between Y₁ and Y₂, resulting in the formation of a π bond, but the bond is far away from the benzene ring on the right side, does not conjugate with the benzene ring, and has deep energy level, which are beneficial to hole injection; when Y₃ is a heteroatom, its HOMO energy level can be further deepened, and hole injection can be increased, making the compound more suitable as a hole injection material, a hole transport material or an electron block material.
In an exemplary embodiment of the present disclosure, the structural formula of the arylamine compound may be:

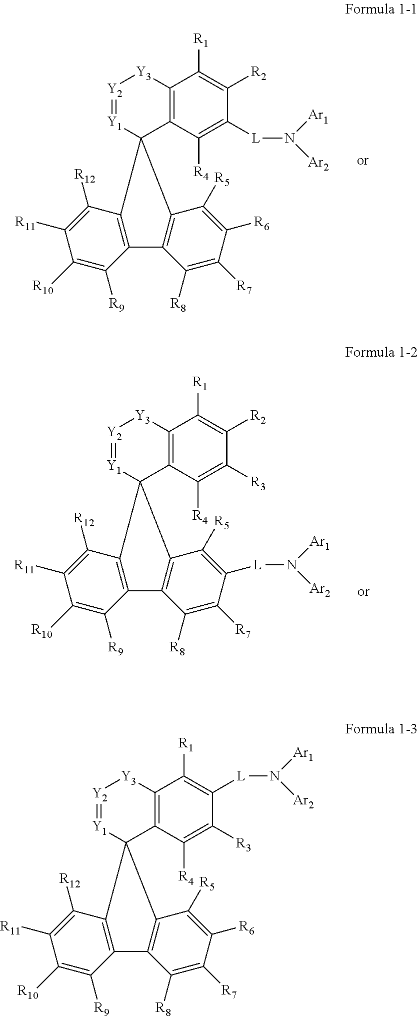

wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

In an exemplary embodiment of the present disclosure, the structural formula of the arylamine compound may be:

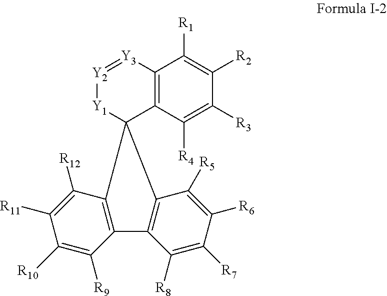

wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R1 to R12 and R₁₅ to R₁₇ are the same as those in Formula I.

In the compound represented by Formula I-2, a double bond is formed between Y₂ and Y₃, resulting in the formation of a bond, and the bond overlaps with π orbits of benzene rings to a large extent and have better conjugation, so the compound represented by Formula I-2 have relatively high hole transport ability; when Y₁ is a heteroatom, its HOMO energy level can become deeper, which can increase the injection of positive charge, and make the compound more suitable as a hole injection material, a hole transport material or an electron block material.

In an exemplary embodiment of the present disclosure, the structural formula of the arylamine compound may be:

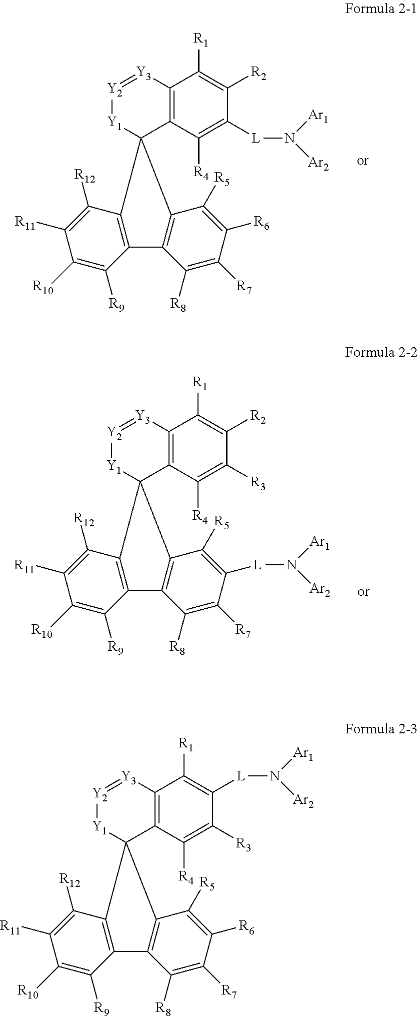

wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R₁ to R₁₂ and R₁₅ to R₁₇ are the same as those in Formula I.

In an exemplary embodiment of the present disclosure, the structural formula of the arylamine compound may be:

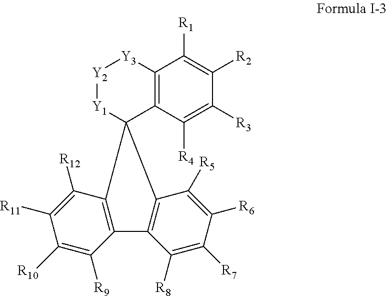

wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

In the compound represented by Formula I-3, σ bonds are formed between Y₁ and Y₂, and between Y₂ and Y₃. Since the bond length of a σ bond is longer than that of a π bond, the compound represented by Formula I-3 have greater spatial stereoscopic property and smaller transverse current; when Y₁ is a heteroatom, its HOMO energy level can become deeper, which can increase the injection of positive charge, and make the compound more suitable as a hole injection material, a hole transport material or an electron block material.

When -L-N(Ar₁)-Ar₂ is contained in the compound represented by formula I and an arylamino group is contained in Ar₁ or Ar₂, the compound represented by Formula I is a diarylamine compound, which can obtain stronger hole property, and the HOMO energy level becomes shallow, which is beneficial to the generation of holes. At this time, the compound is more suitable as a hole transport material.

In an exemplary embodiment of the present disclosure, the structural formula of the arylamine compound may be:

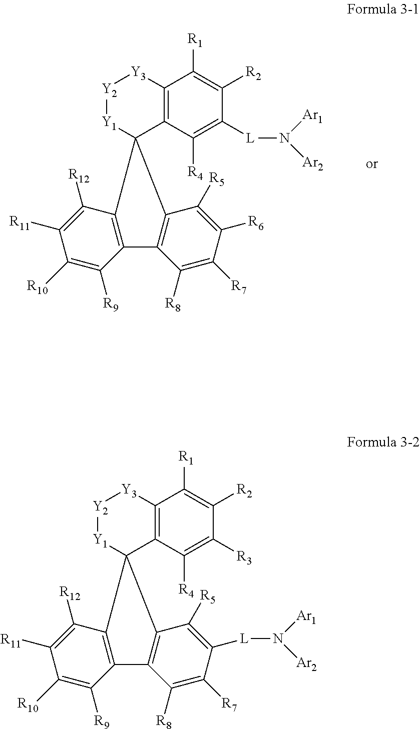

wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

In an exemplary embodiment of the present disclosure, in Formula I-3, Formula 3-1, and Formula 3-2, Y₁, Y₂, and Y₃ are not N at the same time. For example, Y₁ and Y₃ are N, and Y₂ is not N.

In an exemplary embodiment of the present disclosure, Ar₁ and Ar₂ may be each independently selected from any one of a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar₁ and Ar₂ and the N connected with Ar₁ and Ar₂ can form a ring.

In an exemplary embodiment of the present disclosure, Ar₁ and Ar₂ may be an alkyl group with 4 to 39 carbon atoms, for example, an alkyl group with 4 to 12, 12 to 16, 16 to 28, 28 to 39 carbon atoms.

In an exemplary embodiment of the present disclosure, Ar₁ and Ar₂ may be each independently selected from any one of the following groups:

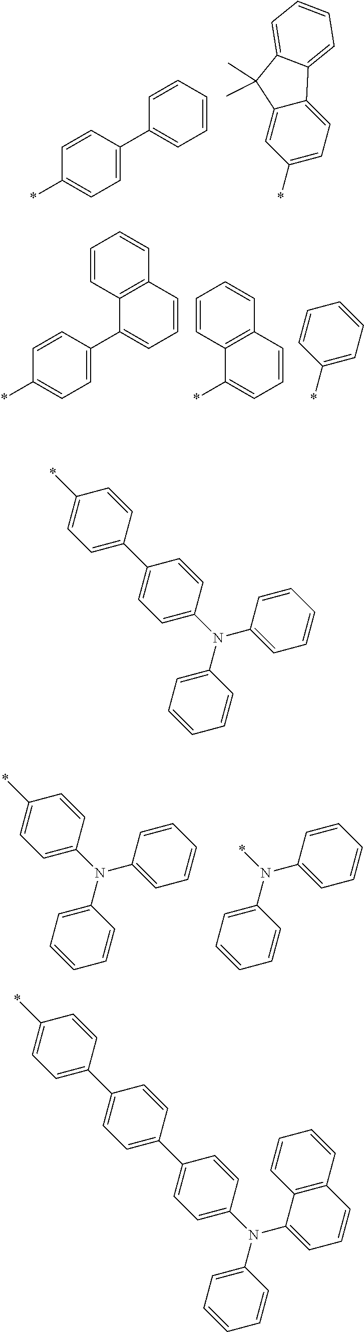

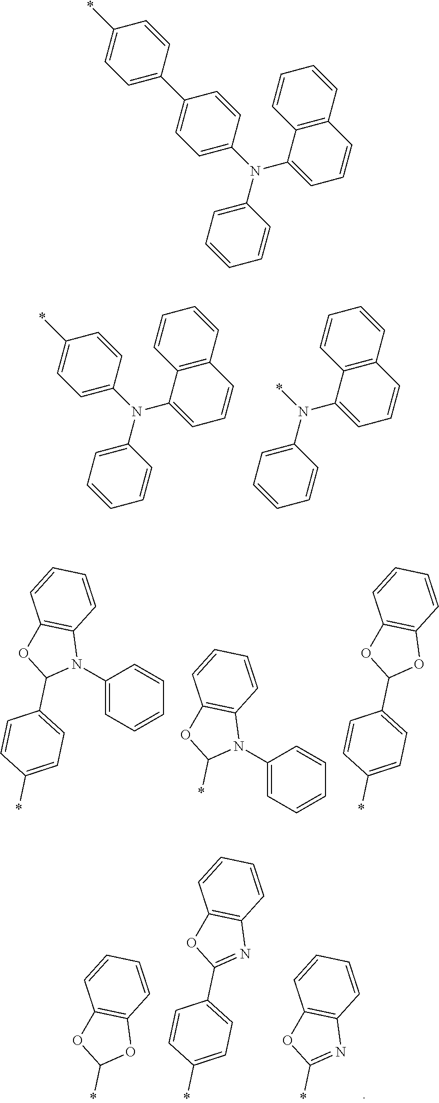

In an exemplary embodiment of the present disclosure, R₁₃ to R₁₈ may be each independently selected from any one of hydrogen, deuterium, an alkyl group with 1 to 4 carbon atoms, and an aryl group with 6 to 20 carbon atoms.

In an exemplary embodiment of the present disclosure, R₁ to R₁₂ that are not -L-N(Ar₁)-Ar₂ may be independently selected from any one of hydrogen and deuterium.

In an exemplary embodiment of the present disclosure, L may be selected from any one of a single bond, a phenylene group, a benzene or phenyl substituted phenylene group, and a biphenyl substituted phenylene group.

In an exemplary embodiment of the present disclosure, the arylamine compound may include any one of the following compounds:

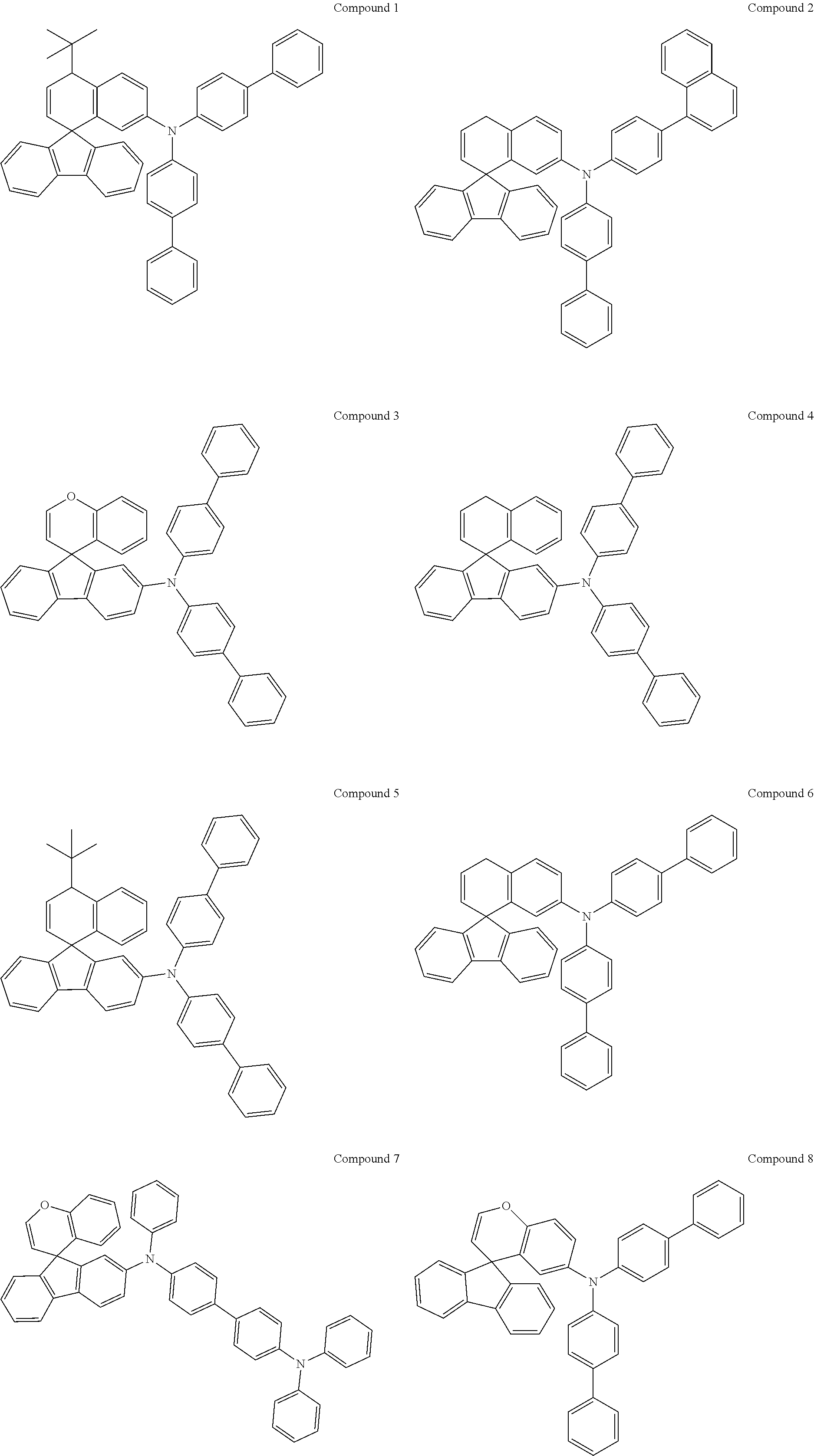

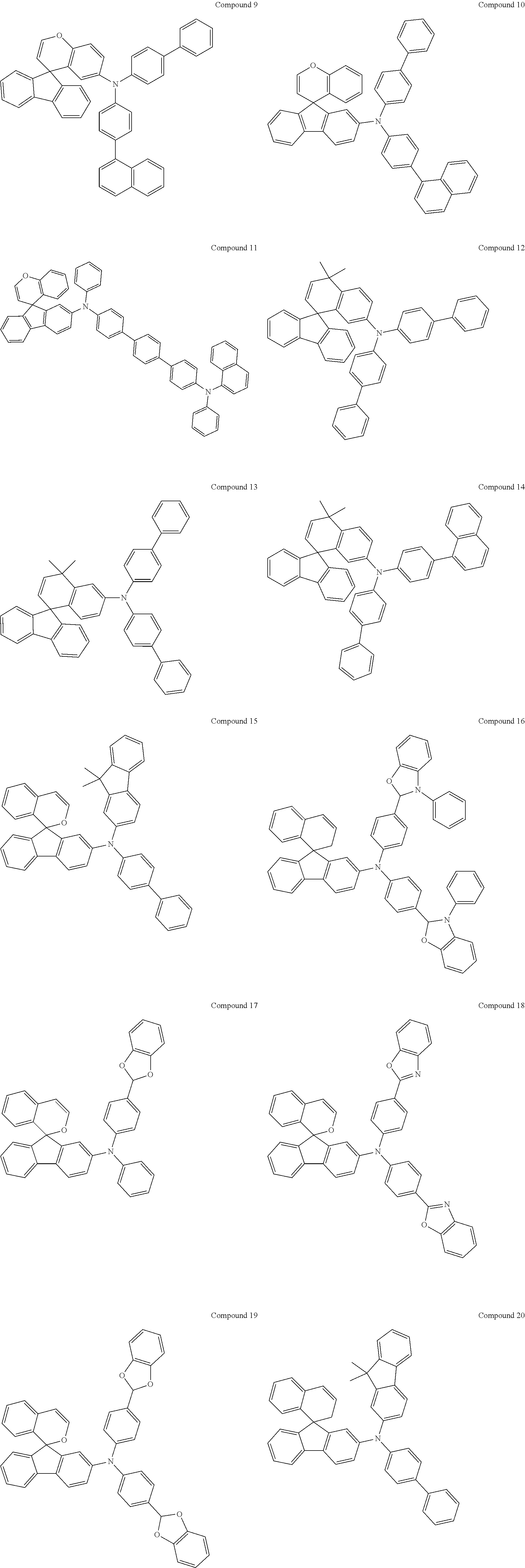

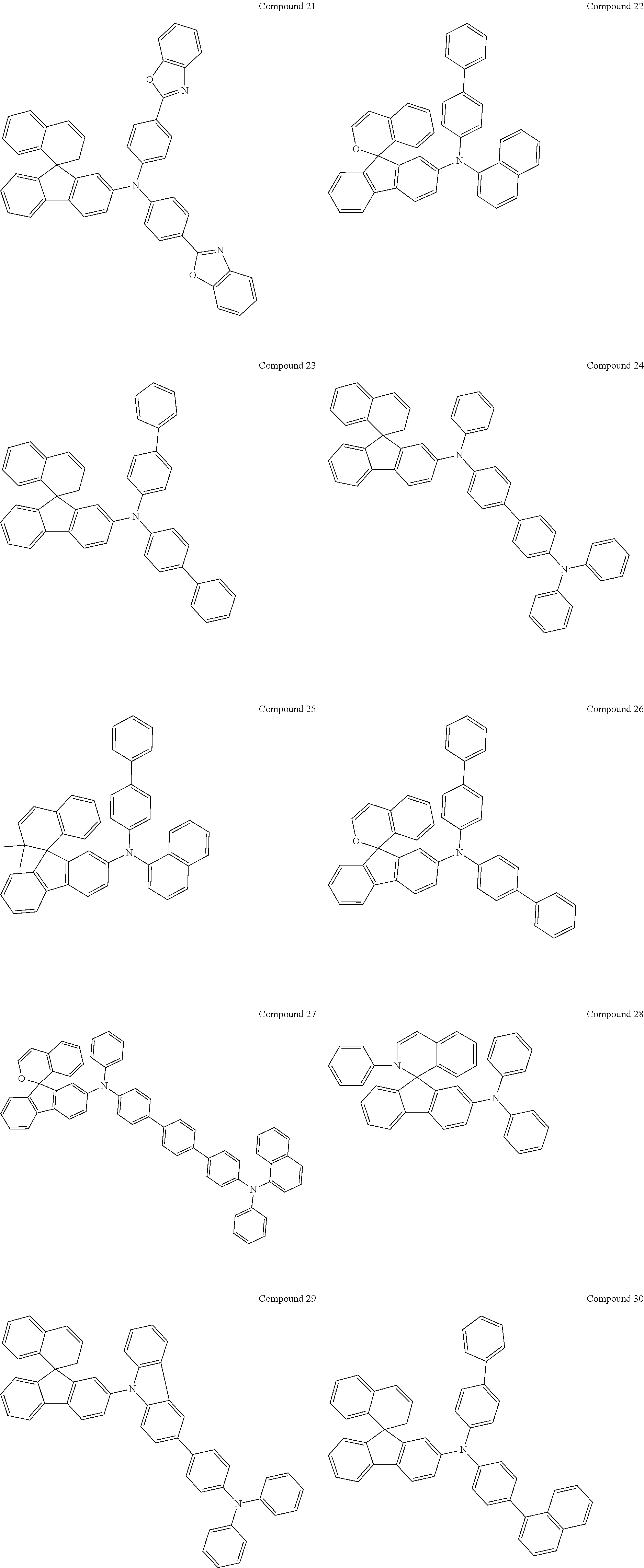

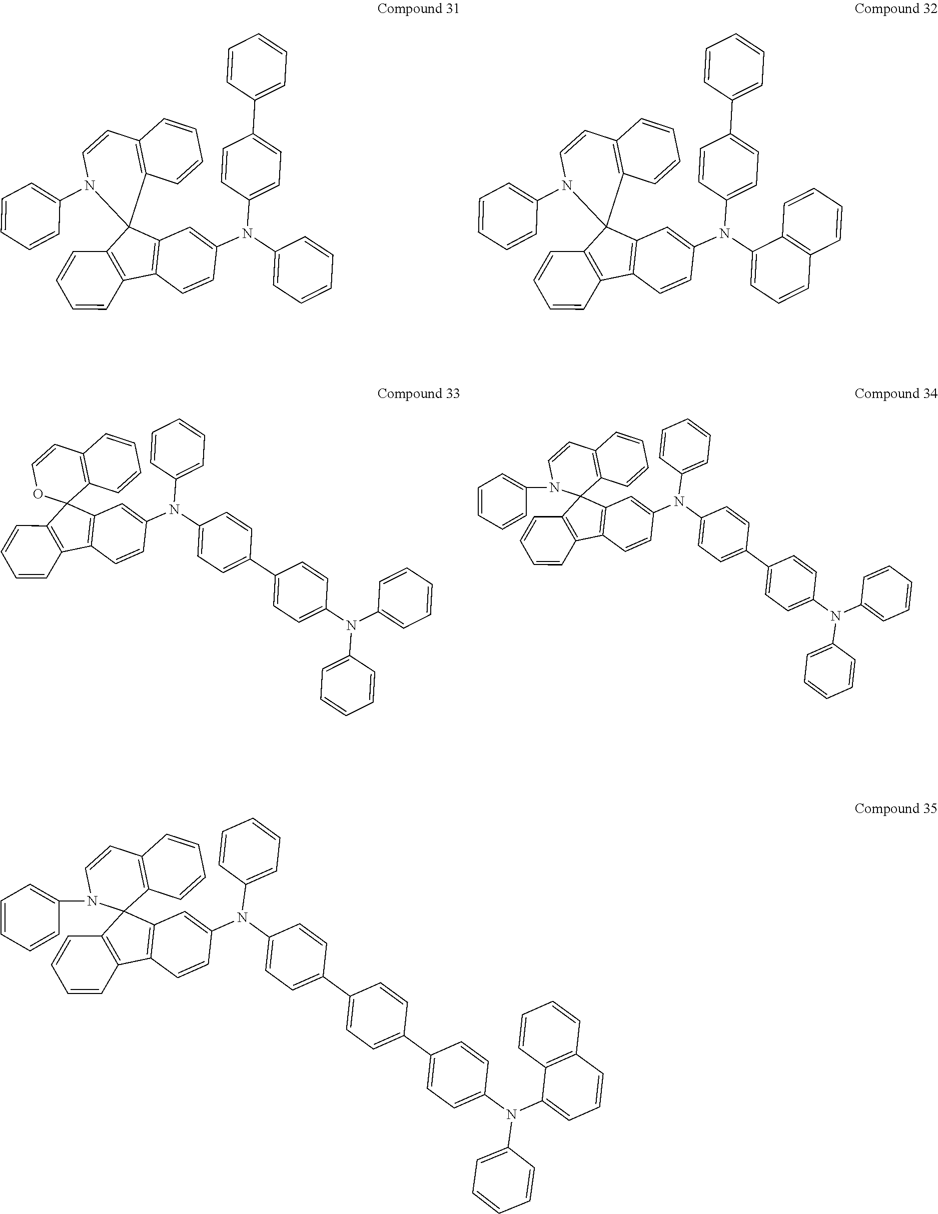 [SMILES: C1=CN(c2ccccc2)C2(c3ccccc31)c1ccccc1-c1ccc(N(c3ccccc3)c3ccc(-c4ccccc4)cc3)cc12 ; C1=CN(c2ccccc2)C2(c3ccccc31)c1ccccc1-c1ccc(N(c3ccc(-c4ccccc4)cc3)c3cccc4ccccc34)cc12 ; C1=Cc2ccccc2C2(O1)c1ccccc1-c1ccc(N(c3ccccc3)c3ccc(-c4ccc(N(c5ccccc5)c5ccccc5)cc4)cc3)cc12 ; C1=CN(c2ccccc2)C2(c3ccccc31)c1ccccc1-c1ccc(N(c3ccccc3)c3ccc(-c4ccc(N(c5ccccc5)c5ccccc5)cc4)cc3)cc12 ; C1=CN(c2ccccc2)C2(c3ccccc31)c1ccccc1-c1ccc(N(c3ccccc3)c3ccc(-c4ccc(-c5ccc(N(c6ccccc6)c6cccc7ccccc67)cc5)cc4)cc3)cc12]

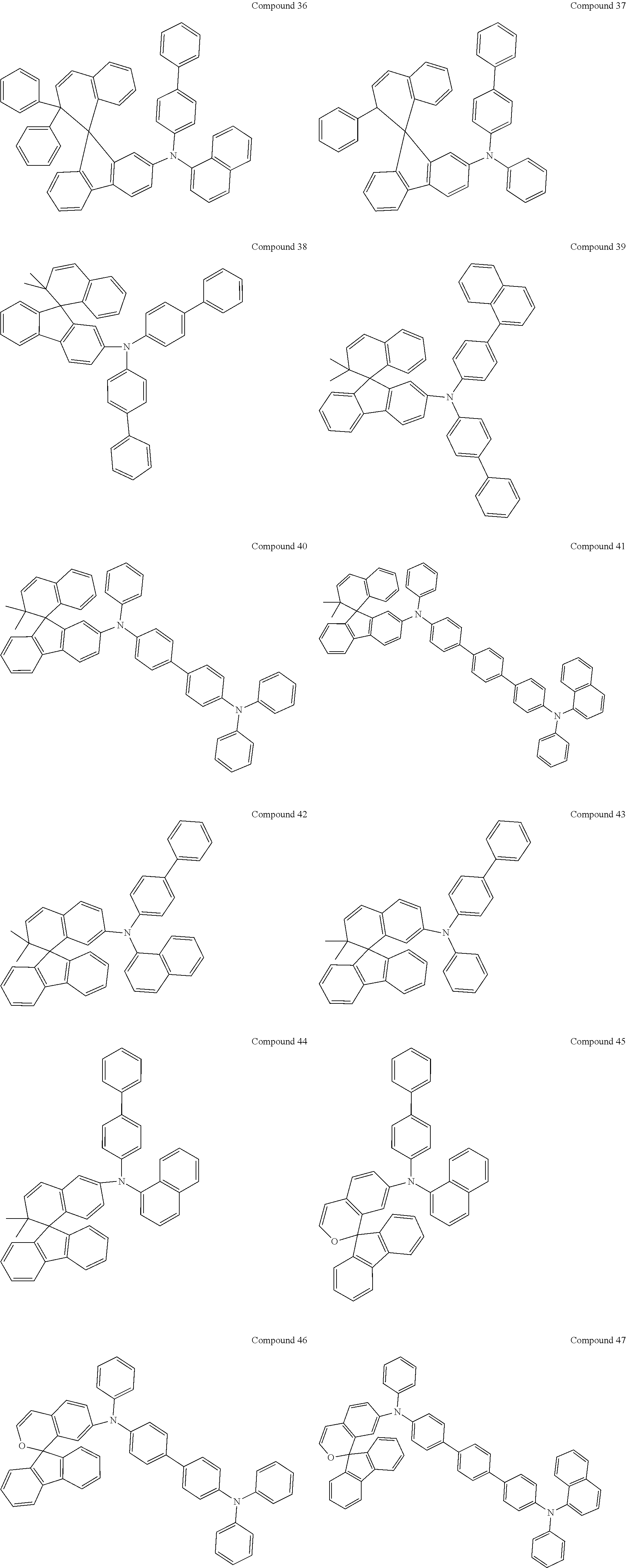

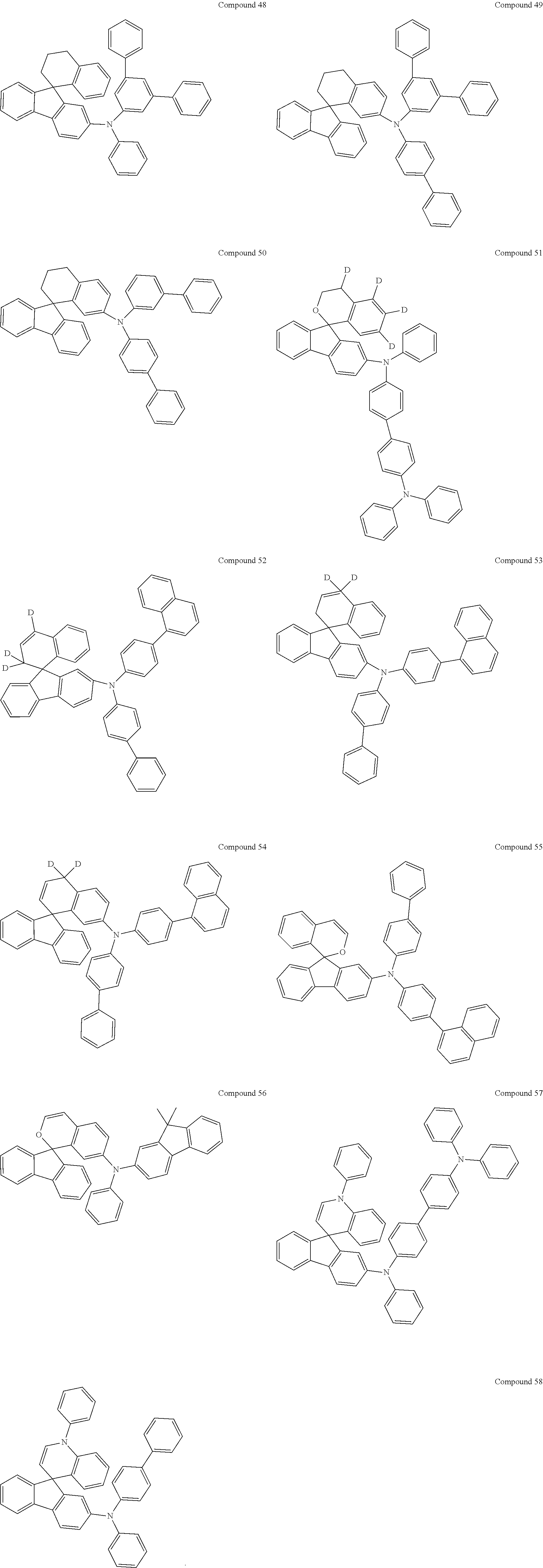

In an exemplary embodiment of the present disclosure, the glass transition temperature of the arylamine compound may be ≥110° C., for example, it may be 120° C. to 180° C., and for another example, it may be about 120° C., 125° C., 130° C., 135° C., 140° C., 145° C., 150° C., 155° C., 160° C., 165° C., 170° C., 175° C., or 180° C.

In an exemplary embodiment of the present disclosure, the Highest Occupied Molecular Orbital (HOMO) energy level of the arylamine compound may be −4.6 eV to −5.7 eV, for example, it may be −4.6 eV, −4.7 eV, −4.8 eV, −4.9 eV, −5.0 eV, −5.1 eV, −5.2 eV, −5.3 eV, −5.4 eV, −5.5 eV, −5.6 eV, or −5.7 eV; and for another example, it may be −4.6 eV to −4.9 eV, or −5.3 eV to −5.5 eV. In an exemplary embodiment of the present disclosure, the triplet energy level of the arylamine compound may be 1.8 eV to 2.6 eV, for example, it may be 1.8 eV, 1.9 eV, 2.0 eV, 2.1 eV, 2.2 eV, 2.3 eV, 2.4 eV, 2.5 eV, or 2.6 eV; and for another example, it may be 2.1 eV to 2.6 eV, and for another example, it may be 2.4 eV to 2.6 eV.

An embodiment of the present disclosure further provides use of the arylamine compound as described above as a hole injection material.

In an exemplary embodiment of the present disclosure, the arylamine compound as described above can be used as a host material of a hole injection layer of an organic electroluminescent device.

The arylamine compound of embodiments of the present disclosure still has relatively large transverse resistance after P-doping. When the doping is 5%, the transverse current is only 100 times of that when no doping, while the current material is only doped 1%, and the transverse current is increased to nearly 600 times. Therefore, the arylamine compound of embodiments of the present disclosure can be used as a host material of a hole injection layer of an OLED device, and can effectively reduce the color crosstalk and solve the difficulties encountered by OLED device manufacturers at present.

An embodiment of the present disclosure further provides use of the arylamine compound as described above as a hole transport material.

The arylamine compound of embodiments of the present disclosure has suitable HOMO energy levels, chemical stability, and relatively high charge mobility, and thus can be used as a hole transport material. The use of an organic electroluminescent device comprising a hole transport layer formed from an arylamine compound of embodiments of the present disclosure can obtain characteristics of high efficiency, low voltage, and long lifetime.

An embodiment of the present disclosure further provides use of the arylamine compound as described above as an electron block material.

The arylamine compound of embodiments of the present disclosure has higher triplet energy level and electrical stability, and can prevent triplet exciton overflow. The use of an organic electroluminescent device comprising an electron block layer formed from an arylamine compound of embodiments of the present disclosure can obtain characteristics of high efficiency, and long lifetime.

An embodiment of the present disclosure further provides an electroluminescent device, including the arylamine compound as described above.

In an exemplary embodiment of the present disclosure, the electroluminescent device may include a hole injection layer, a hole transport layer and an electron block layer.

In an exemplary embodiment of the present disclosure, the hole injection layer and the hole transport layer each comprise the arylamine compound as described above.

In an exemplary embodiment of the present disclosure, the hole injection layer and the electron block layer each comprise the arylamine compound as described above.

In an exemplary embodiment of the present disclosure, the hole injection layer, the hole transport layer, and the electron block layer each comprise the arylamine compound as described above.

When the arylamine compound disclosed in embodiments of the present disclosure is used as two or three of the host material of the hole injection layer, the material of the hole transport layer and the material of the electron block layer of the same device, the use of evaporation sources can be reduced and the production cost can be reduced.

In an exemplary embodiment of the present disclosure, the electroluminescent device may include: an anode, a hole injection layer, a hole transport layer, an electron block layer, an emitting layer, a hole block layer, an electron transport layer, an electron injection layer (EIL), a cathode, and a light extraction layer.

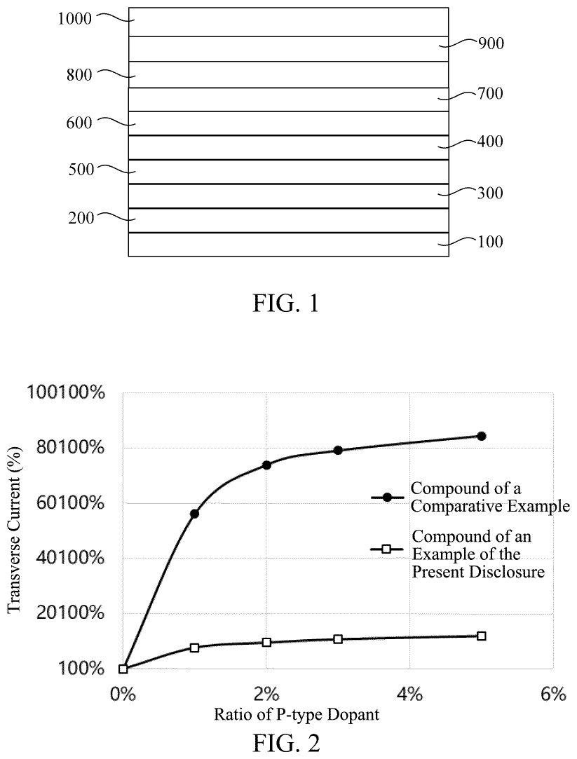

FIG. 1 is a schematic diagram of structure of an electroluminescent device according to an exemplary embodiment of the present disclosure. As shown in FIG. 1, the electroluminescent device may include: an anode 100, a hole injection layer 200, a hole transport layer 300, an electron block layer 400, an emitting layer 500, a hole block layer 600, an electron transport layer 700, an electron injection layer 800, a cathode 900 and a light extraction layer 1000. The hole injection layer 200 is disposed on a surface on one side of the anode 100, the hole transport layer 300 is disposed on a surface of the hole injection layer 200 on a side away from the anode 100, the electron block layer 400 is disposed on a surface of the hole transport layer 300 on a side away from the anode 100, the emitting layer 500 is disposed on a surface of the electron block layer 400 on a side away from the anode 100, the hole block layer 600 is disposed on a surface of the emitting layer 500 on a side away from the anode 100, the electron transport layer 700 is disposed on a surface of the hole block layer 600 on a side away from the anode 100, the electron injection layer 800 is disposed on a surface of the electron transport layer 700 on a side away from the anode 100, the cathode 900 is disposed on a surface of the electron injection layer 800 on a side away from the anode 100, and the light extraction layer 1000 is disposed on a surface of the cathode 900 on a side away from the anode 100.

In an exemplary embodiment of the present disclosure, the anode may be a material with a high work function. For example, for a bottom emission device, the anode may adopt a transparent oxide material, such as indium tin oxide (ITO) or Indium Zinc Oxide (IZO). Alternatively, for a top emission device, the anode may adopt a composite structure of a metal and a transparent oxide, such as Ag/ITO (Indium Tin Oxide), Ag/IZO (Indium Zinc Oxide), Al/ITO, Al/IZO or ITO/Ag/ITO and the like, which may ensure good reflectivity.

In an exemplary embodiment of the present disclosure, the material of the hole injection layer may include the arylamine compound provided by embodiments of the present disclosure, and transition metal oxides, for example, may include any one or more of molybdenum oxides, titanium oxides, vanadium oxides, rhenium oxides, ruthenium oxides, chromium oxides, zirconium oxides, hafnium oxides, tantalum oxides, silver oxides, tungsten oxides, manganese oxides.

In another exemplary embodiment of the present disclosure, the material of the hole injection layer may include a p-type dopant of a strong electron absorption system and a hole transport material.

The p-type dopant may include any one or more of 2,3,6,7,10,11-hexocyano-1,4,5,8,9,12-hexazabenzophenanthrene, 2,3,5,6-tetrafluoro-7,7′,8,8′-tetracyano-p-benzoquinone (F4TCNQ), 1,2,3-tris[(cyano)(4-cyano-2,3,5,6-tetrafluorophenyl)methylene]cyclopropane.

The hole transport material may include any one or more of an arylamine compound provided by embodiments of the present disclosure, an arylamine-based hole transport material, a dimethylfluorene-based hole transport material, and a carbazole-based hole transport material; for example, the hole transport material may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); 9-phenyl-3-[4-(10-phenyl-9-anthryl)phenyl]-9H-carbazole (PCzPA).

In an exemplary embodiment of the present disclosure, the hole injection layer may be formed by evaporation.

In an exemplary embodiment of the present disclosure, a material of the hole transport layer may include any one or more of an arylamine compound provided by embodiments of the present disclosure, an arylamine-based hole transport material, a dimethylfluorene-based hole transport material, and a carbazole-based hole transport material; for example, the material of the hole transport layer may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); and 9-phenyl-3-[4-(10-phenyl-9-anthryl)phenyl]-9H-carbazole (PCzPA).

In an exemplary embodiment of the present disclosure, the hole transport layer may be formed by evaporation.

In an exemplary embodiment of the present disclosure, the material of the electron block layer may include any one or more of an arylamine compound provided by embodiments of the present disclosure, an arylamine-based electron block material, a dimethylfluorene-based electron block material, and a carbazole-based electron block material; for example, the material of the electron block layer may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); 9-phenyl-3-[4-(10-phenyl-9-anthryl)phenyl]-9H-carbazole (PCzPA).

In an exemplary embodiment of the present disclosure, the electron block layer may be formed by evaporation.

In an exemplary embodiment of the present disclosure, a material of the emitting layer may include one luminescent material, or may include two or more luminescent materials. For example, a host luminescent material and a guest luminescent material doped into the host luminescent material may be included.

In an exemplary embodiment of the present disclosure, the electroluminescent device may be a blue electroluminescent device, a green electroluminescent device, or a red electroluminescent device, a material of the emitting layer of the blue electroluminescent device includes a blue luminescent material, a material of the emitting layer of the green electroluminescent device includes a green luminescent material, and a material of the emitting layer of the red electroluminescent device may include a red luminescent material.

In an exemplary embodiment of the present disclosure, the blue luminescent material may include any one or more of a pyrene derivative-based blue luminescent material, an anthracene derivative-based blue luminescent material, a fluorene derivative-based blue luminescent material, a perylene derivative-based blue luminescent material, a styrylamine derivative-based blue luminescent material, and a metal complex-based blue luminescent material.

For example, the blue luminescent material may include any one or more of N1,N6-bis([1,1′-biphenyl]-2-yl)-N1,N6-bis([1,1′-biphenyl]-4-yl) pyrene-1,6-diamine; 9,10-di-(2-naphthyl)anthracene (ADN); 2-methyl-9,10-di-2-naphthyl anthracene (MADN); 2,5,8,11-tetra-tert-butylperylene (TBPe); 4,4′-bis[4-(diphenylamino)styryl]biphenyl (BDAVBi); 4,4′-bis[4-(di-p-tolylamino)styryl]biphenyl (DPAVBi); and bis(4,6-difluorophenylpyridine-C2,N)pyridine formyl iridium (FIrpic).

In an exemplary embodiment of the present disclosure, the green luminescent material may include any one or more of a coumarin dye, a quinacridine copper derivative-based green luminescent material, a polycyclic aromatic hydrocarbon-based green luminescent material, a diamine anthracene derivative-based green luminescent material, a carbazole derivative-based green luminescent material, and a metal complex-based green luminescent material.

For example, the green luminescent material may include coumarin 6 (C-6); coumarin 545T (C-525T); quinacridine copper (QA); N,N′-dimethylquinacridone (DMQA); 5,12-diphenylnaphthonaphthalene (DPT); N10,N10′-diphenyl-N10,N10′-dibenzoyl-9,9′-dianthracene-10,10′-diamine (BA-NPB); tris(8-hydroxyquinoline) aluminum (III) (Alq3), tris(2-phenylpyridine)iridium(Ir(ppy)3); and acetylpyruvate bis(2-phenylpyridine)iridium (Ir(ppy)2(acac)).

In an exemplary embodiment of the present disclosure, the red luminescent material may include any one or more of a DCM-based red luminescent material and a metal complex-based red luminescent material.

For example, the red luminescent material may include any one or more of 4-(dicyanomethylene)-2-methyl-6-(4-dimethylaminostyryl)-4H-pyran (DCM); 4-(dicyanomethylene)-2-tert-butyl-6-(1,1,7,7-tetramethyljulolidyl-9-enyl)-4H-pyran (DCJTB); di(1-phenylisoquinoline)(acetylacetone)iridium(III) (Ir(piq)2(acac)); octaethylporphyrin platinum (PtOEP); di(2-(2′-benzothiophenyl)pyridine-N,C3′)(acetylacetone)iridium (Ir(btp)2(acac)).

In an exemplary embodiment of the present disclosure, the emitting layer may be formed by evaporation.

In an exemplary embodiment of the present disclosure, the material of the hole block layer may include an aromatic heterocyclic-based hole block material, for example, may include any one or more of hole block materials based on benzimidazole and its derivatives, hole block materials based on imidazopyridine and its derivatives, hole block materials based on benzimidazophenanthridine derivatives, hole block materials based on pyrimidine and its derivatives, hole block materials based on triazine derivatives, hole block materials based on pyridine and its derivatives, hole block materials based on pyrazine and its derivatives, hole block materials based on quinoxaline and its derivatives, hole block materials based on oxadiazole and its derivatives, hole block materials based on quinoline and its derivatives, hole block materials based on isoquinoline derivatives, hole block materials based on phenanthroline derivatives, hole block materials based on diazaphosphorole, hole block materials based on phosphine oxide, hole block materials based on aromatic ketone, lactams, and hole block materials based on boranes.

For another example, the material of the hole block layer may include any one or more of 2-(4-biphenyl)-5-(4-tert-butylphenyl)-1,3,4-oxadiazole(PBD); 1,3-bis[5-(p-tert-butylphenyl)-1,3,4-oxadiazole-2-yl]benzene(OXD-7); 3-(4-tert-butylphenyl)-4-phenyl-5-(4-biphenyl)-1,2,4-triazole(TAZ); 3-(4-tert-butylphenyl)-4-(4-ethylphenyl)-5-(4-biphenyl)-1,2,4-triazole(p-EtTAZ); red phenanthroline (BPhen); (BCP); 4,4′-bis(5-methylbenzoxazol-2-yl)stilbene (BzOs).

In an exemplary embodiment of the present disclosure, the hole block layer may be formed by evaporation.

In an exemplary embodiment of the present disclosure, the material of the electron transport layer may include aromatic heterocyclic-based electron transport materials, for example, may include any one or more of electron transport materials based on benzimidazole and its derivatives, electron transport materials based on imidazopyridine and its derivatives, electron transport materials based on benzimidazolophenanthridine derivatives, electron transport materials based on pyrimidine and its derivatives, electron transport materials based on triazine derivatives, electron transport materials based on pyridine and its derivatives, electron transport materials based on pyrazine and its derivatives, electron transport materials based on quinoxaline and its derivatives, electron transport materials based on oxadiazole and its derivatives, electron transport materials based on quinoline and its derivatives, electron transport materials based on isoquinoline derivatives, electron transport materials based on phenanthroline derivatives, electron transport materials based on diazaphosphorole, electron transport materials based on phosphine oxide, electron transport materials based on aromatic ketone, lactams, electron transport materials based on boranes.

For another example, the material of the electron transport layer may include any one or more of 2-(4-biphenyl)-5-(4-tert-butylphenyl)-1,3,4-oxadiazole(PBD); 1,3-bis[5-(p-tert-butylphenyl)-1,3,4-oxadiazole-2-yl]benzene (OXD-7); 3-(4-tert-butylphenyl)-4-phenyl-5-(4-biphenyl)-1,2,4-triazole (TAZ); 3-(4-tert-butylphenyl)-4-(4-ethylphenyl)-5-(4-biphenyl)-1,2,4-triazole(p-EtTAZ); red phenanthroline(BPhen); (BCP); 4,4′-bis(5-methylbenzoxazol-2-yl)stilbene (BzOs).

In an exemplary embodiment of the present disclosure, the electron transport layer may be formed by evaporation.

In an exemplary embodiment of the present disclosure, the material of the electron injection layer may include any one or more of an alkali metal electron injection material and a metal electron injection material.

For example, the material of the electron injection layer may include any one or more of LiF, Yb, Mg, and Ca.

In an exemplary embodiment of the present disclosure, the electron injection layer may be formed by evaporation.

In an exemplary embodiment of the present disclosure, the cathode may be formed by the metal with relatively low work function, such as Al, Ag, and Mg, or formed by an alloy containing a metal material with low work function.

In an exemplary embodiment of the present disclosure, the light extraction layer may be formed by evaporation and using a diarylamine-based light extraction material.

An embodiment of the present disclosure further provides a display apparatus, including the electroluminescent device as described above.

In an exemplary embodiment, the display apparatus may include a plurality of electroluminescent devices. For example, the electroluminescent device may be a blue electroluminescent device, a green electroluminescent device, or a red electroluminescent device, and the display apparatus may include a blue electroluminescent device, a green electroluminescent device, and a red electroluminescent device.

The display apparatus may be any product or component with a display function, such as a mobile phone, a tablet computer, a television, a display, a laptop computer, a digital photo frame, a navigator, a vehicle-mounted display, a smart watch, and a smart bracelet.

Embodiments of the present disclosure further provide a synthesis method for the arylamine compound as described above, comprising the following steps:

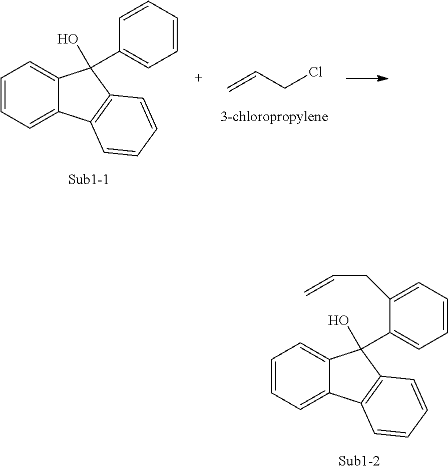

A compound Sub1-1, 3-chloropropylene and AlCl₃ were added respectively in a 500 ml round bottom flask under nitrogen atmosphere. A compound Sub1-2 was obtained after the reaction (yield: 60%).

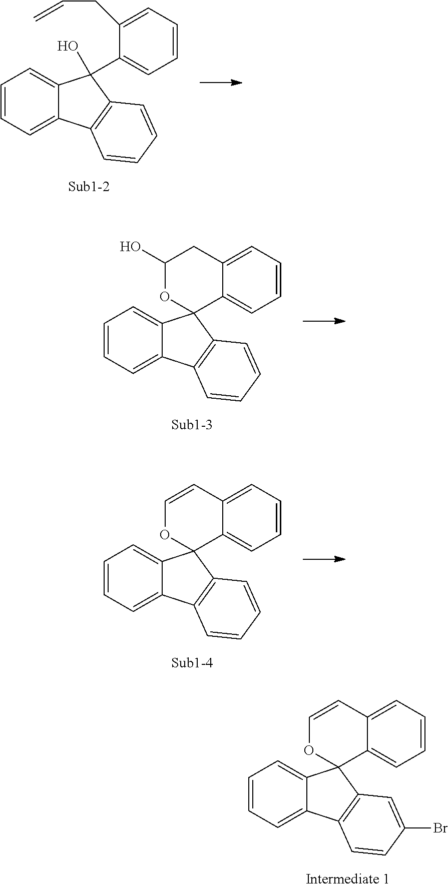 [SMILES: C=CCc1ccccc1C1(O)c2ccccc2-c2ccccc21 ; OC1Cc2ccccc2C2(O1)c1ccccc1-c1ccccc12 ; C1=Cc2ccccc2C2(O1)c1ccccc1-c1ccccc12 ; Brc1ccc2c(c1)C1(OC=Cc3ccccc31)c1ccccc1-2]

Sub1-2 obtained above was dissolved in ethyl ether. Under the action of n-butyl lithium, Cu₂(CN)₂, and N,N,N′,N′-tetramethylethylenediamine, Sub1-3 was obtained after the reaction (yield: 65%). After adding concentrated sulfuric acid and heating the reaction for 4 hours, Sub1-4 was obtained. Then hot HBr was added for substitution reaction. The solvent was removed. Intermediate 1 was obtained from the crude product with column chromatography on silica gel.

The obtained Intermediate 1 was then used to synthesize the arylamine compound of embodiments of the present disclosure. C—C coupling reaction, Suzuki coupling reaction, Negishi coupling reaction, Yamamoto coupling reaction, Grignard Cross coupling reaction, Stille coupling reaction, Heck coupling reaction, C—N coupling reaction, Buchwald coupling reaction, Ullmann coupling reaction, silylation reaction, phosphorization reaction, boronation reaction, polycondensation reaction, and the like could be used for the synthesis method.

The general reaction formula is as follows:

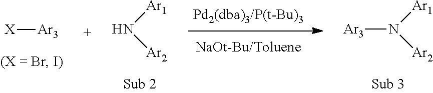

Below are synthesis processes and performance tests and comparisons of arylamine compounds according to some exemplary embodiments of the present disclosure.

### Synthesis of Compounds

The reaction materials used in Synthesis Examples 1 to 10 were shown in Table 1.

TABLE 1

| Synthesis | | | |
|---|---|---|---|
| Examples | Reactant 1 | Reactant 2 | Compound |
| 1 | 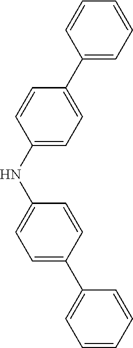 [SMILES: c1ccc(-c2ccc(Nc3ccc(-c4ccccc4)cc3)cc2)cc1] | 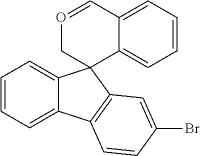 Intermediate 1 | 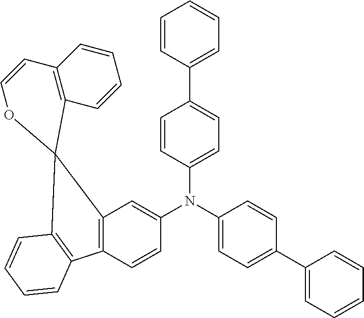 [SMILES: C1=Cc2ccccc2C2(O1)c1ccccc1-c1ccc(N(c3ccc(-c4ccccc4)cc3)c3ccc(-c4ccccc4)cc3)cc12] Compound 26 |
| 2 | 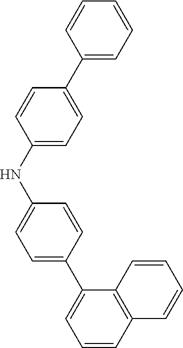 [SMILES: c1ccc(-c2ccc(Nc3ccc(-c4cccc5ccccc45)cc3)cc2)cc1] | | 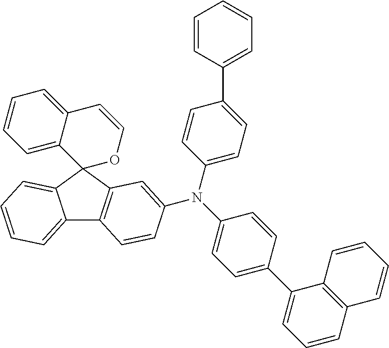 [SMILES: C1=Cc2ccccc2C2(O1)c1ccccc1-c1ccc(N(c3ccc(-c4ccccc4)cc3)c3ccc(-c4cccc5ccccc45)cc3)cc12] |
| | | | Compound 55 |
| 3 | 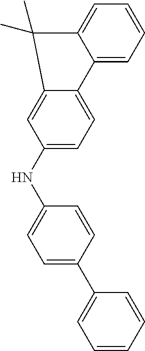 [SMILES: CC1(C)c2ccccc2-c2ccc(Nc3ccc(-c4ccccc4)cc3)cc21] | | 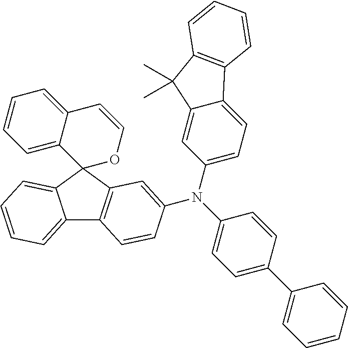 [SMILES: CC1(C)c2ccccc2-c2ccc(N(c3ccc(-c4ccccc4)cc3)c3ccc4c(c3)C3(OC=Cc5ccccc53)c3ccccc3-4)cc21] |
| | | | Compound 15 |
| 4 | 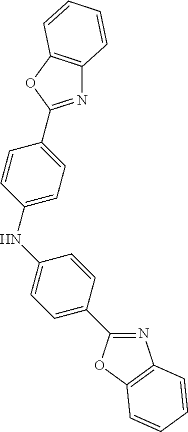 [SMILES: c1ccc2oc(-c3ccc(Nc4ccc(-c5nc6ccccc6o5)cc4)cc3)nc2c1] | | 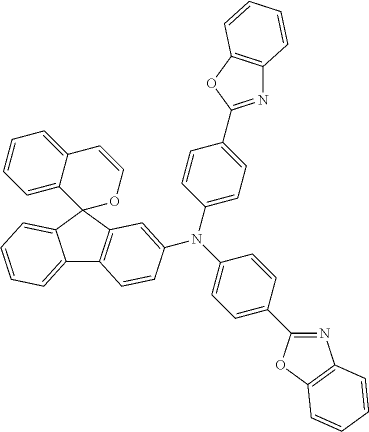 [SMILES: C1=Cc2ccccc2C2(O1)c1ccccc1-c1ccc(N(c3ccc(-c4nc5ccccc5o4)cc3)c3ccc(-c4nc5ccccc5o4)cc3)cc12] |
| | | | Compound 18 |
| 5 | 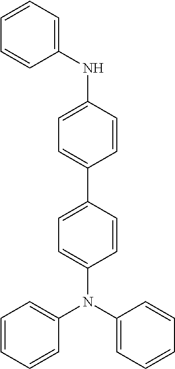 [SMILES: c1ccc(Nc2ccc(-c3ccc(N(c4ccccc4)c4ccccc4)cc3)cc2)cc1] | | 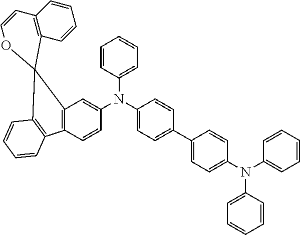 [SMILES: C1=Cc2ccccc2C2(O1)c1ccccc1-c1ccc(N(c3ccccc3)c3ccc(-c4ccc(N(c5ccccc5)c5ccccc5)cc4)cc3)cc12] Compound 33 |
| 6 | 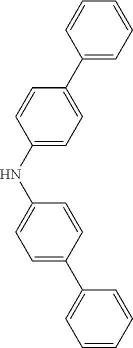 [SMILES: c1ccc(-c2ccc(Nc3ccc(-c4ccccc4)cc3)cc2)cc1] | 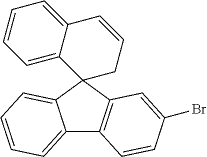 [SMILES: Brc1ccc2c(c1)C1(CC=Cc3ccccc31)c1ccccc1-2] | 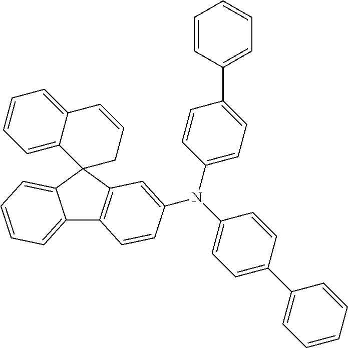 [SMILES: C1=Cc2ccccc2C2(C1)c1ccccc1-c1ccc(N(c3ccc(-c4ccccc4)cc3)c3ccc(-c4ccccc4)cc3)cc12] |
| | | | Compound 23 |
| 7 | 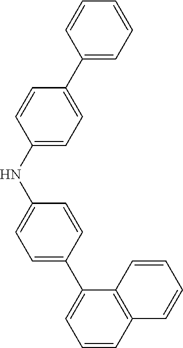 [SMILES: c1ccc(-c2ccc(Nc3ccc(-c4cccc5ccccc45)cc3)cc2)cc1] | | 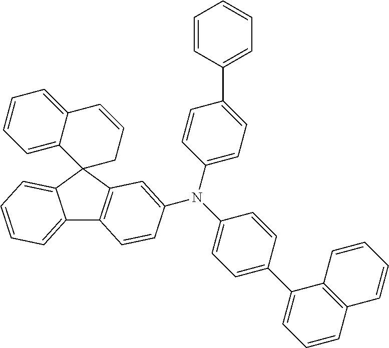 [SMILES: C1=Cc2ccccc2C2(C1)c1ccccc1-c1ccc(N(c3ccc(-c4ccccc4)cc3)c3ccc(-c4cccc5ccccc45)cc3)cc12] |
| | | | Compound 30 |
| 8 | 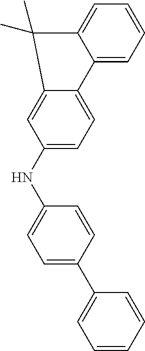 [SMILES: CC1(C)c2ccccc2-c2ccc(Nc3ccc(-c4ccccc4)cc3)cc21] | | 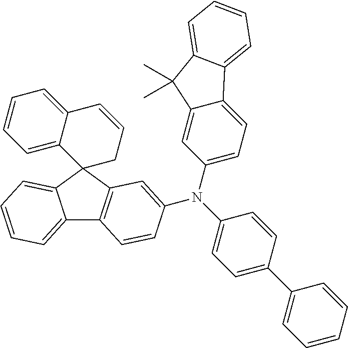 [SMILES: CC1(C)c2ccccc2-c2ccc(N(c3ccc(-c4ccccc4)cc3)c3ccc4c(c3)C3(CC=Cc5ccccc53)c3ccccc3-4)cc21] |
| | | | Compound 20 |
| 9 | 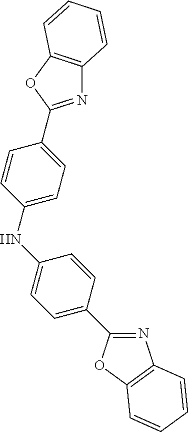 [SMILES: c1ccc2oc(-c3ccc(Nc4ccc(-c5nc6ccccc6o5)cc4)cc3)nc2c1] | | 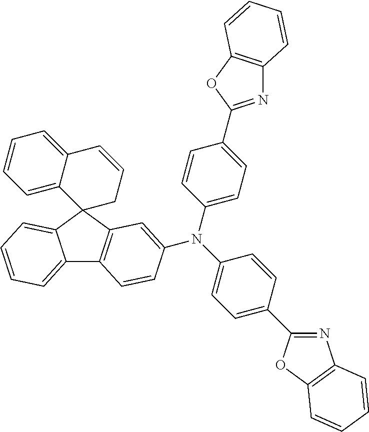 [SMILES: C1=Cc2ccccc2C2(C1)c1ccccc1-c1ccc(N(c3ccc(-c4nc5ccccc5o4)cc3)c3ccc(-c4nc5ccccc5o4)cc3)cc12] |
| | | | Compound 21 |
| 10 | 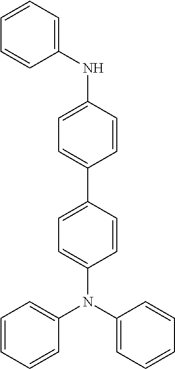 [SMILES: c1ccc(Nc2ccc(-c3ccc(N(c4ccccc4)c4ccccc4)cc3)cc2)cc1] | | 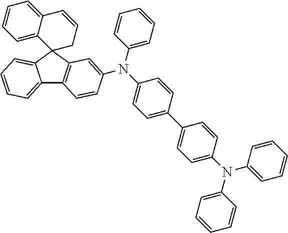 [SMILES: C1=Cc2ccccc2C2(C1)c1ccccc1-c1ccc(N(c3ccccc3)c3ccc(-c4ccc(N(c5ccccc5)c5ccccc5)cc4)cc3)cc12] Compound 24 |

The synthesis processes of the compounds of Synthesis Examples 1 to 10 were similar to each other. Taking the synthesis process of Compound 15 of Synthesis Example 3 as an example, the synthesis route of the compound was as follows:

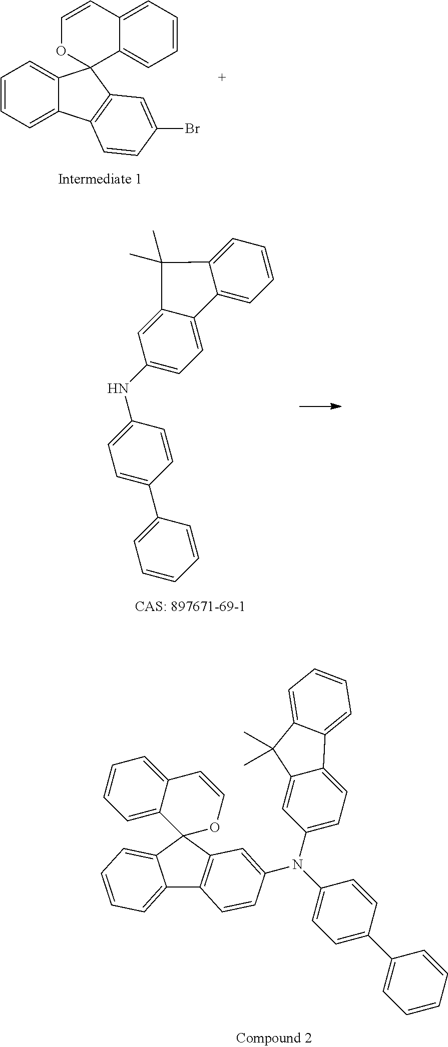 [SMILES: Brc1ccc2c(c1)C1(OC=Cc3ccccc31)c1ccccc1-2 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc4c(c3)C3(OC=Cc5ccccc53)c3ccccc3-4)cc21]

The Intermediate 1 (11.2 mmol) and N-[1,1′-biphenyl-4-yl]-9,9-dimethyl-9H-fluoren-2-amine [897671-69-1] (14.00 mmol) were completely dissolved in 200 ml of tetrahydrofuran in a 500 ml round bottom flask under nitrogen atmosphere, followed by the addition of a 2 M aqueous potassium carbonate solution (100 ml). After the addition of bis(tri-tert-butyl phosphine) palladium (0.23 g, 0.20 mmol), the reaction was heated and stirred for 2 hours. The temperature was reduced to room temperature, and the aqueous layer was removed. After drying with anhydrous magnesium sulfate, concentrating under reduced pressure and recrystallizing with 200 ml of ethyl acetate were carried out to give compound 15 (4.67 g, 65% yield).

### Structural Characterization Data of Compound 15:

MS m/z: 641.27 (100.0%).

Element content (%): C₄₈H₃₅NO, C, 89.83; H, 5.50; N, 2.18; O, 2.49.

¹H NMR (500 MHz, CDCl₃): δ 1.69 (6H, s), 5.87 (1H, d), 6.56 (1H, d), 7.12-7.55 (21H, m), 7.70-7.76 (2H, d), 7.80-7.90 (4H, dd).

### Structural Characterization Data of Compound 18:

MS m/z: 683.22 (100.0%).

Element content (%): C₄₇H₂₉N₃O₃, C, 82.56%; H, 4.28%; N, 6.15%; O, 7.02%.

¹H NMR (500 MHz, CDCl₃): δ 6.05 (1H, d), 6.85 (1H, d), 7.12-7.28 (4H, m), 7.30-7.90 (23H, m).

### Structural Characterization Data of Compound 20:

MS m/z: 639.12 (100.0%).

Element content (%): C₄₉H₃₇N, C, 91.98%; H, 5.83%; N, 2.19%.

¹H NMR (500 MHz, CDCl₃): δ 1.45 (6H, s), 2.8-2.99 (2H, dd), 5.87 (1H, d), 6.76 (1H, d), 7.02-7.90 (27H, m).

### Structural Characterization Data of Compound 26:

MS m/z: 601.65 (100.0%).

Element content (%): C₄₅H₃₁NO, C, 89.82%; H, 5.19%; N, 2.33%; O, 2.66%.

¹H NMR (500 MHz, CDCl₃): δ 5.95 (1H, d), 6.84 (1H, d), 7.12-7.28 (4H, m), 7.30-7.90 (25H, m).

### Structural Characterization Data of Compound 30:

MS m/z: 649.2770 (100.0%).

Element content (%): C₅₀H₃₅N, C, 92.42%; H, 5.43%; N, 2.16%.

¹H NMR (500 MHz, CDCl₃): δ 2.8-2.99 (2H, dd), 5.87 (1H, d), 6.76 (1H, d), 7.02-8.01 (31H, m).

The performance test data of some compounds in the present application were shown in Table 2:

TABLE 2

| | | HOMO | Triplet Energy |
|---|---|---|---|
| Compound | Tg/° C. | Energy Level/eV | Level/eV |
| Compound 2 | 148 | −5.38 | 2.36 |
| Compound 15 | 143 | −5.33 | 2.25 |
| Compound 20 | 138 | −5.37 | 2.31 |
| Compound 26 | 140 | −5.51 | 2.25 |
| Compound 31 | 139 | −5.44 | 2.18 |
| Compound 45 | 135 | −5.28 | 2.55 |
| Compound 56 | 145 | −5.41 | / |
| Compound 57 | / | −5.23 | / |
| Compound 58 | / | −5.34 | / |

The transverse current of the compounds prepared in the above Examples was measured with the structure of the device shown in FIG. 1. When 3% P-type dopant F4TCNQ was doped, the corresponding currents at a voltage of 10V were shown in Table 3.

TABLE 3

| Compound | Transverse Current |
|---|---|
| Comparative | 100.0% |
| Example 1 (NPB) | |
| Comparative | 83.0% |
| Example 2 (EB-1) | |
| Compound 1 | 12.9% |
| Compound 2 | 11.5% |
| Compound 3 | 13.0% |
| Compound 4 | 13.6% |
| Compound 5 | 14.2% |
| Compound 6 | 12.3% |
| Compound 7 | 10.3% |
| Compound 8 | 10.7% |
| Compound 9 | 10.5% |
| Compound 10 | 11.3% |
| Compound 11 | 10.1% |
| Compound 15 | 13.9% |
| Compound 16 | 9.6% |
| Compound 17 | 8.7% |
| Compound 18 | 8.90% |
| Compound 19 | 7.80% |
| Compound 20 | 11.3% |
| Compound 21 | 11.1% |
| Compound 22 | 10.6% |
| Compound 25 | 11.4% |
| Compound 40 | 10.3% |
| Compound 41 | 10.6% |

FIG. 2 is curves of transverse current of the compound (NPB) of Comparative Example 1 versus compound 2 of an example of the present disclosure as a function of a doping ratio of a P-type dopant.

It could be seen that the arylamine compounds prepared in the Examples of the present disclosure had a small transverse current after doping the P-type dopant. Therefore, the color crosstalk of the device could be effectively suppressed.

### Application of the Compounds in Devices

The arylamine compounds of Examples of the present disclosure was used as the host material of a hole injection layer or the hole transport material to prepare an OLED device, and the structural formula of other materials used in the device was as follows:

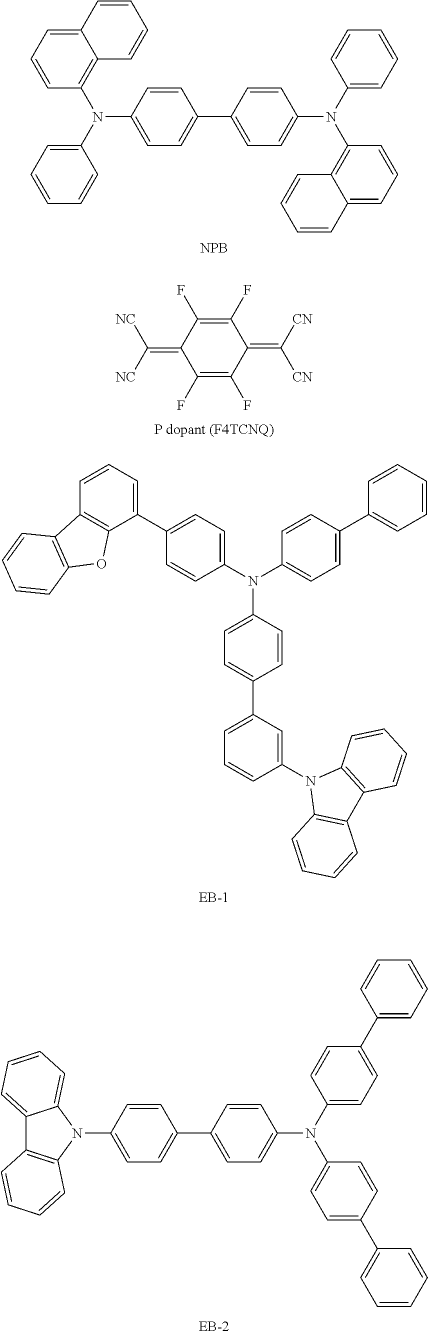 [SMILES: c1ccc(N(c2ccc(-c3ccc(N(c4ccccc4)c4cccc5ccccc45)cc3)cc2)c2cccc3ccccc23)cc1 ; "NPB" ; N#CC(C#N)=c1c(F)c(F)c(=C(C#N)C#N)c(F)c1F ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

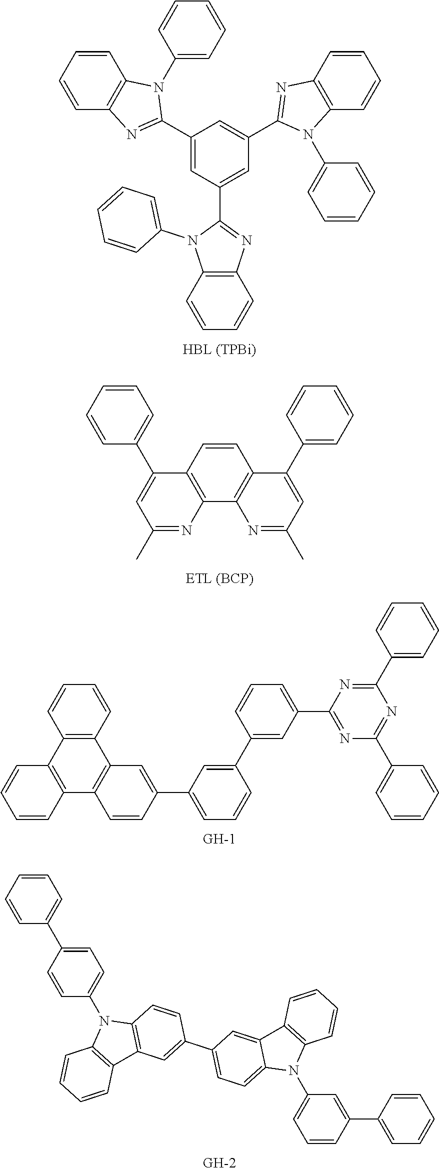 [SMILES: c1ccc(-n2c(-c3cc(-c4nc5ccccc5n4-c4ccccc4)cc(-c4nc5ccccc5n4-c4ccccc4)c3)nc3ccccc32)cc1 ; Cc1cc(-c2ccccc2)c2ccc3c(-c4ccccc4)cc(C)nc3c2n1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

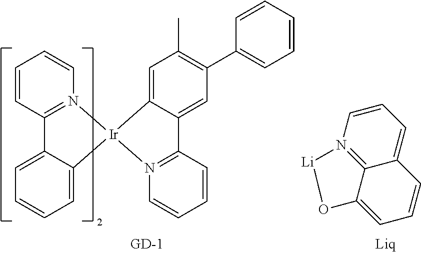

The film structure of a red light device was shown in Table 4, in which the arylamine compounds of Examples of the present disclosure were used as the host material of a hole injection layer or the hole transport material. The performance of devices was shown in Table 5.

TABLE 4

| Device | | | | | | | |
|---|---|---|---|---|---|---|---|
| Structure | HI | HT | Prime | EML | HB | ET | EI |
| Comparative | NPB: P dopant | NPB | EB-2 | BH:BD | TPBI | BCP:Liq = 1:1 | LIF |
| Example 1 | 3% 10 nm | 80 nm | | (1%, | (5 nm) | | 1 nm |
| Device | Compound 2: | Compound | | 20 nm) | | | |
| Example 1-1 | P dopant 3% | 2 | | | | | |
| | 10 nm | 80 nm | | | | | |
| Device | Compound 40: | Compound | | | | | |
| Example 1-2 | P dopant 3% | 40 | | | | | |
| | 10 nm | 80 nm | | | | | |
| Device | Compound 45: | Compound | | | | | |
| Example 1-3 | P dopant 3% | 45 | | | | | |
| | 10 nm | 80 nm | | | | | |

TABLE 5
Performance of Red Light Devices

| | | | Lifetime |
|---|---|---|---|
| | Voltage | Efficiency | (LT95 @ 15 mA/cm²) |
| Comparative Example 1 | 100% | 100% | 100% |
| Example 1-1 | 97% | 123% | 125% |
| Example 1-2 | 96% | 135% | 158% |
| Example 1-3 | 97% | 134% | 150% |

The film structure of a blue light device was shown in Table 6, in which the arylamine compounds of Examples of the present disclosure were used as the host material of a hole injection layer and the electron block layer (blue light emitting auxiliary layer) material. The performance of devices was shown in Table 7.

TABLE 6

| Device | | | | | | | |
|---|---|---|---|---|---|---|---|
| Structure | HI | HT | EBL | EML | HB | ET | EI |
| Comparative | NPB: P dopant | NPB | EB-2 | BH: | TPBI | BCP:Liq = 1:1 | LIF |
| Example 2 | 3% 10 nm | 80 nm | 10 nm | BD | | | 1 nm |
| Device | NPB: P dopant | | Compound | (1%, | | | |
| Example 2-1 | 3% 10 nm | | 22 | 20 nm) | | | |
| | | | 10 nm | | | | |
| Device | NPB: P dopant | | Compound | | | | |
| Example 2-2 | 3% 10 nm | | 10 | | | | |
| | | | 10 nm | | | | |
| Device | Compound 5: P | | EB-2 | | | | |
| Example 2-3 | dopant | | 10 nm | | | | |
| | 3% 10 nm | | | | | | |
| Device | Compound 22: | | Compound | | | | |
| Example 2-4 | P dopant | | 22 | | | | |
| | 3% 10 nm | | 10 nm | | | | |
| Device | Compound 10: | | Compound | | | | |
| Example 2-5 | P dopant | | 10 | | | | |
| | 3% 10 nm | | 10 nm | | | | |

TABLE 7
Performance of Blue Light Devices

| | | | Lifetime |
|---|---|---|---|
| | Voltage | Efficiency | (LT95 @ 15 mA/cm²) |
| Comparative Example 2 | 100% | 100% | 100% |
| Example 2-1 | 96% | 122% | 122% |
| Example 2-2 | 97% | 123% | 137% |
| Example 2-3 | 100% | 102% | 109% |
| Example 2-4 | 96% | 129% | 132% |
| Example 2-5 | 96% | 131% | 143% |

The film structure of a green light device was shown in Table 8, in which the arylamine compounds of Examples of the present disclosure were used as the host material of a hole injection layer and the electron block layer (green light emitting auxiliary layer) material. The performance of devices was shown in Table 9.

TABLE 8

| Device | | | | | | | |
|---|---|---|---|---|---|---|---|
| Structure | HI | HT | EBL | EML | HB | ET | EI |
| Comparative | NPB: P | NPB | EB-1 | GH-1: | TPBI | BCP:Liq = 1:1 | LIF |
| Example 3 | dopant | 80 nm | 10 nm | GH-2: | | | 1 nm |
| | 3% 10 nm | | | GD | | | |
| Device | Compound 17: | | Compound | | | | |
| Example 3-1 | P dopant | | 17 | | | | |
| | 3% 10 nm | | 3% 10 nm | | | | |
| Device | Compound 18: | | Compound | | | | |
| Example 3-2 | P dopant | | 18 | | | | |
| | 3% 10 nm | | 3% 10 nm | | | | |

TABLE 9
Performance of Green Light Devices

| | | | Lifetime |
|---|---|---|---|
| | Voltage | Efficiency | (LT95 @ 15 mA/cm²) |
| Comparative Example 3 | 100% | 100% | 100% |
| Example 3-1 | 96% | 122% | 132% |
| Example 3-2 | 97% | 131% | 147% |

As could be seen from Device Examples 1-1 to 1-3, the arylamine compounds of Examples of the present disclosure could be used as a hole transport material, could improve device efficiency, and enabled the devices to have a longer lifetime and a low working voltage.

As could be seen from Device Examples 2-1, Example 2-2 and Comparative Example 2, the arylamine compounds of Examples of the present disclosure could be used as the material of an electron block layer of a blue light device, improving the efficiency of the device and prolonging the lifetime thereof.

As could be seen from Device Examples 3-1, Example 3-2 and Comparative Example 3, the arylamine compounds of Examples of the present disclosure could be used as the material of an electron block layer of a green light device, improving the efficiency of the device and prolonging the lifetime thereof.

As could be seen from Device Examples 1-1 to 1-3, 2-3 to 2-5, and 3-1 to 3-2, the arylamine compounds of Examples of the present disclosure could be used as the host material of a hole injection layer. The arylamine compounds of Examples of the present disclosure had a relatively low transverse current (transverse current data was shown in Table 1), which could avoid the color crosstalk caused by transverse charge flow during color display, thereby greatly improving the color display effect.

In order to avoid introducing an extra evaporation source during the actual production of the devices, the arylamine compounds of the Examples of the present disclosure could be used as an electron block layer (the light emitting auxiliary layer) and the host material of a hole injection layer at the same time. The evaporation source was not increased, and at the same time, the hole transport material with high mobility was combined (the arylamine compounds of Examples of the present disclosure or the hole transport material commonly used at present could be used), thus achieving the purpose of saving cost and realizing the optimization of display effect.

Although the implementations of the present disclosure are disclosed above, the contents are only implementations adopted to easily understand the present disclosure and not intended to limit the present disclosure. Any skilled person in the art to which the present disclosure pertains may make any modifications and alterations in forms and details of implementation without departing from the spirit and scope of the present disclosure. However, the patent protection scope of the present disclosure should be subject to the scope defined by the appended claims.

1. An arylamine compound, having a general structural formula of: 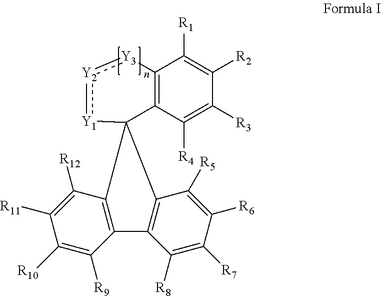
wherein, n is 0, 1 or 2;
at least one of R₁ to R₁₂ is -L-N(Ar₁)-Ar₂, and the R₁ to R₁₂ that are not -L-N(Ar₁)-Ar₂ are each independently selected from any one of hydrogen, deuterium, an alkyl group with 1 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group;
Ar₁ and Ar₂ are each independently selected from any one of an alkyl group with 4 to 39 carbon atoms, a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted cycloalkyl group with 3 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted cycloalkyl group with 3 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar₁ and Ar₂ and the N connected with Ar₁ and Ar₂ form a ring;
L is selected from any one of a single bond, a substituted or unsubstituted arylene group with 6 to 39 carbon atoms, and a substituted or unsubstituted heteroarylene group with 5 to 60 carbon atoms; wherein, the substituted arylene group with 6 to 39 carbon atoms and the substituted heteroarylene group with 5 to 60 carbon atoms mean being substituted by an aryl group with 6 to 39 carbon atoms and a heteroaryl group with 5 to 60 carbon atoms;
Y₁, Y₂, and Y₃ satisfy one of the following conditions:
1) the dotted line between Y₁ and Y₂ exists, and a double bond is formed between Y₁ and Y₂; and the dotted line between Y₂ and Y₃ does not exist;
Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—;
2) the dotted line between Y₁ and Y₂ does not exist; and the dotted line between Y₂ and Y₃ exists, and a double bond is formed between Y₂ and Y₃;
Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; and
3) the dotted line between Y₁ and Y₂ does not exist, and the dotted line between Y₂ and Y₃ does not exist;
Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—;
R₁₃ to R₁₇ are each independently selected from any one of hydrogen, deuterium, an alkyl group with 1 to 39 carbon atoms, and an aryl group with 6 to 39 carbon atoms.

2. The arylamine compound according to claim 1, wherein Y₁, Y₂, and Y₃ satisfy at least one of the following conditions:
1) at least one of Y₁, Y₂, and Y₃ is selected from —N(R₁₅)—, —O— and S—, and Y₁, Y₂, and Y₃ are not simultaneously selected from —O— and —S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

3. The arylamine compound according to claim 2, wherein Y₁, Y₂, and Y₃ satisfy at least one of the following conditions:
1) one of Y₁, Y₂, and Y₃ is selected from any one of —N(R₁₃)—, —O— and S—;
2) the dotted line between Y₁ and Y₂ exists, and a double bond is formed between Y₁ and Y₂; and the dotted line between Y₂ and Y₃ does not exist; and
3) the dotted line between Y₁ and Y₂ does not exist; and the dotted line between Y₂ and Y₃ exists, and a double bond is formed between Y₂ and Y₃.

4. The arylamine compound according to claim 1, having a structural formula of: 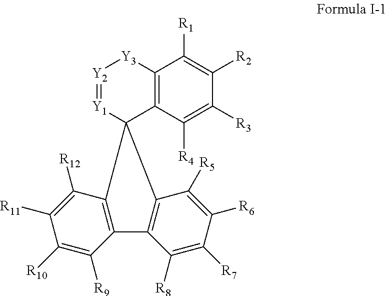
wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

5. The arylamine compound according to claim 4, having a structural formula of: 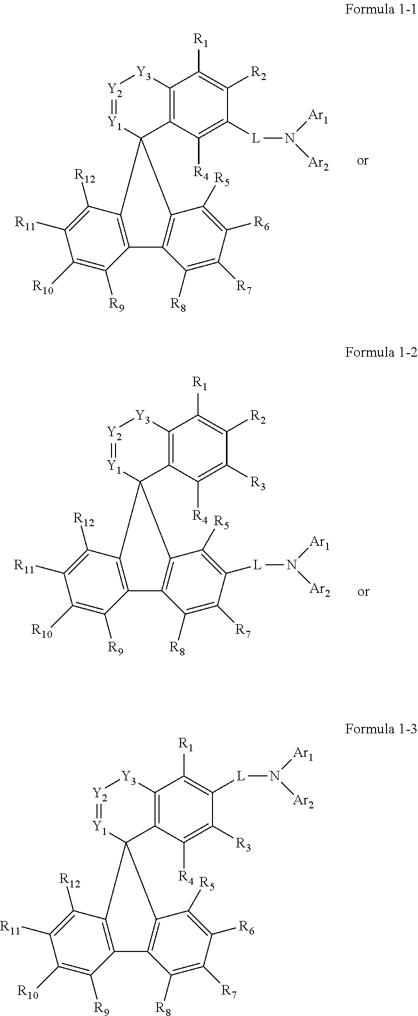
wherein, Y₁ is selected from any one of N and —C(R₁₃)—, Y₂ is —C(R₁₄)—, and Y₃ is selected from any one of —N(R₁₅)—, —O—, —S— and —C(R₁₆)(R₁₇)—, definitions of R₁ to R₁₇ are the same as those in Formula I.

6. The arylamine compound according to claim 1, having a structural formula of: 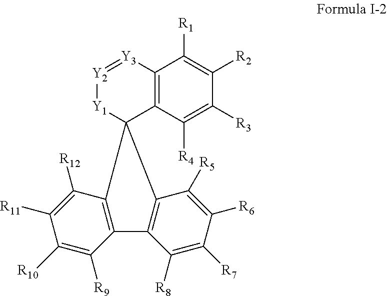
wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R₁ to R₁₂ and R₁₅ to R₁₇ are the same as those in Formula I.

7. The arylamine compound according to claim 6, having a structural formula of: 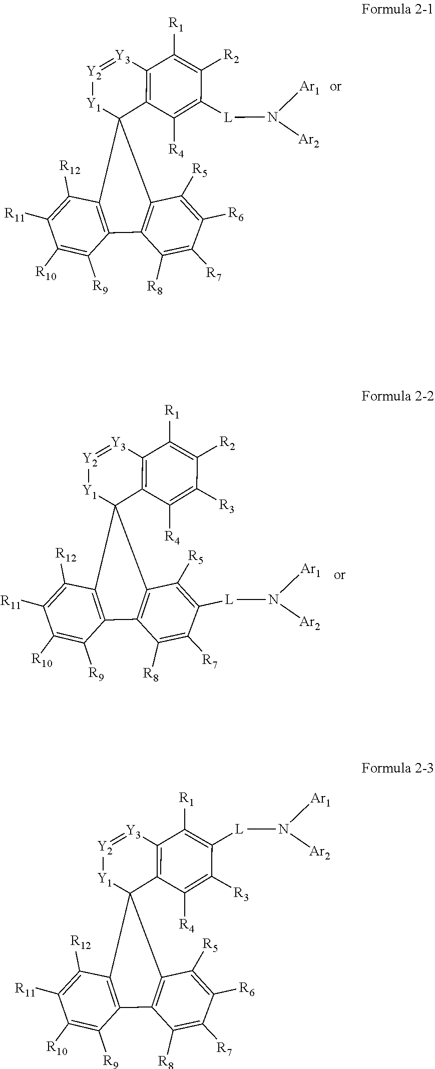
wherein, Y₁ is selected from any one of —N(R₁₅)—, —O—, —S— and —C(R₁₆)(R₁₇)—, Y₂ is —C(R₁₄)—, and Y₃ is selected from any one of N and —C(R₁₃)—; definitions of R₁ to R₁₂ and R₁₅ to R₁₇ are the same as those in Formula I.

8. The arylamine compound according to claim 1, having a structural formula of: 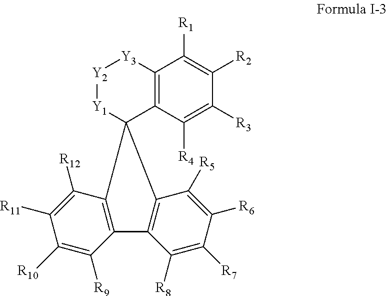
wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

9. The arylamine compound according to claim 8, having a structural formula of: 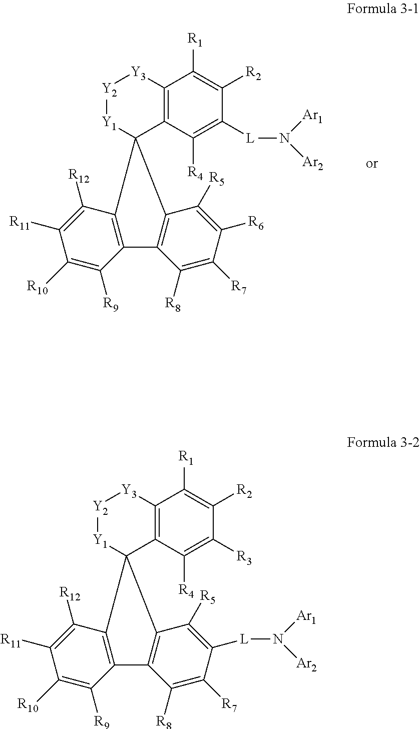
wherein, Y₁, Y₂, and Y₃ are each independently selected from any one of —N(R₁₅)—, —O—, —S— and —C(R₁₆)(R₁₇)—, and Y₁, Y₂, and Y₃ are not simultaneously selected from —O— and —S—; definitions of R₁ to R₁₂ and R₁₅ to R₁₇ are the same as those in Formula I.

10. The arylamine compound according to claim 1, wherein, Ar₁ and Ar₂ are each independently selected from any one of a substituted or unsubstituted aryl group with 6 to 39 carbon atoms, a substituted or unsubstituted heteroaryl group with 5 to 60 carbon atoms, a substituted or unsubstituted aryloxy group with 6 to 60 carbon atoms, a substituted or unsubstituted alkoxy group with 1 to 39 carbon atoms, a substituted or unsubstituted arylamino group with 6 to 39 carbon atoms, a substituted or unsubstituted heterocycloalkyl group with 3 to 39 carbon atoms, a substituted or unsubstituted alkylsilyl group with 1 to 39 carbon atoms, a substituted or unsubstituted alkylboron group with 1 to 39 carbon atoms, a substituted or unsubstituted arylboron group with 6 to 39 carbon atoms, an arylsilyl group with 6 to 39 carbon atoms, a substituted or unsubstituted fluorenyl group, and a substituted or unsubstituted heterofluorenyl group; wherein, the substituted aryl group with 6 to 39 carbon atoms, the substituted heteroaryl group with 5 to 60 carbon atoms, the substituted aryloxy group with 6 to 60 carbon atoms, the substituted alkoxy group with 1 to 39 carbon atoms, the substituted arylamino group with 6 to 39 carbon atoms, the substituted heterocycloalkyl group with 3 to 39 carbon atoms, the substituted alkylsilyl group with 1 to 39 carbon atoms, the substituted alkylboron group with 1 to 39 carbon atoms, the substituted arylboron group with 6 to 39 carbon atoms, the arylsilyl group with 6 to 39 carbon atoms, the substituted fluorenyl group, and the substituted heterofluorenyl group mean being substituted by one or more of the following groups: an alkyl group with 1 to 39 carbon atoms, an aryl group with 6 to 39 carbon atoms, a heteroaryl group with 5 to 60 carbon atoms, an aryloxy group with 6 to 60 carbon atoms, an alkoxy group with 1 to 39 carbon atoms, an arylamino group with 6 to 39 carbon atoms, a cycloalkyl group with 3 to 39 carbon atoms, a heterocycloalkyl group with 3 to 39 carbon atoms, an alkylsilyl group with 1 to 39 carbon atoms, an alkylboron group with 1 to 39 carbon atoms, an arylboron group with 6 to 39 carbon atoms, an arylsilyl group with 6 to 39 carbon atoms, a fluorenyl group, and a heterofluorenyl group; or, the carbons in Ar₁ and Ar₂ and the N connected with Ar₁ and Ar₂ form a ring;

11. The arylamine compound according to claim 10, wherein Ar₁ and Ar₂ are each independently selected from any one of the following groups:

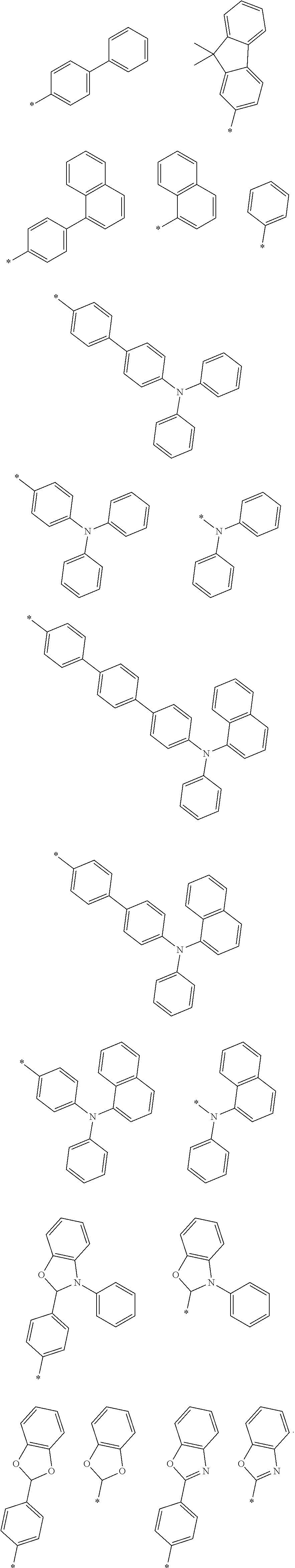

12. The arylamine compound according to claim 1, wherein R₁₃ to R₁₈ are each independently selected from any one of hydrogen, deuterium, an alkyl group with 1 to 4 carbon atoms, and an aryl group with 6 to 20 carbon atoms.

13. The arylamine compound according to claim 1, wherein the R₁ to R₁₂ that are not -L-N(Ar₁)-Ar₂ are each independently selected from any one of hydrogen and deuterium.

14. The arylamine compound according to claim 1, wherein L is selected from any one of a single bond, a phenylene group, a benzene or phenyl substituted phenylene group, and a biphenyl substituted phenylene group.

15. The arylamine compound according to claim 1, comprising any one of the following compounds:

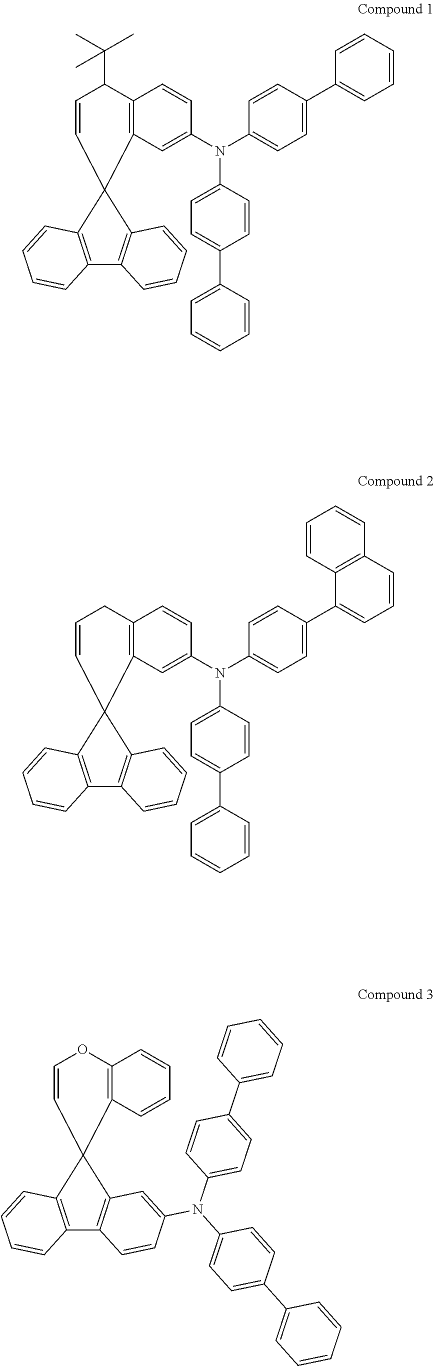 [SMILES: CC(C)(C)C1C=CC2(c3ccccc3-c3ccccc32)c2cc(N(c3ccc(-c4ccccc4)cc3)c3ccc(-c4ccccc4)cc3)ccc21 ; C1=CC2(c3cc(N(c4ccc(-c5ccccc5)cc4)c4ccc(-c5cccc6ccccc56)cc4)ccc3C1)c1ccccc1-c1ccccc12 ; C1=CC2(c3ccccc3O1)c1ccccc1-c1ccc(N(c3ccc(-c4ccccc4)cc3)c3ccc(-c4ccccc4)cc3)cc12]

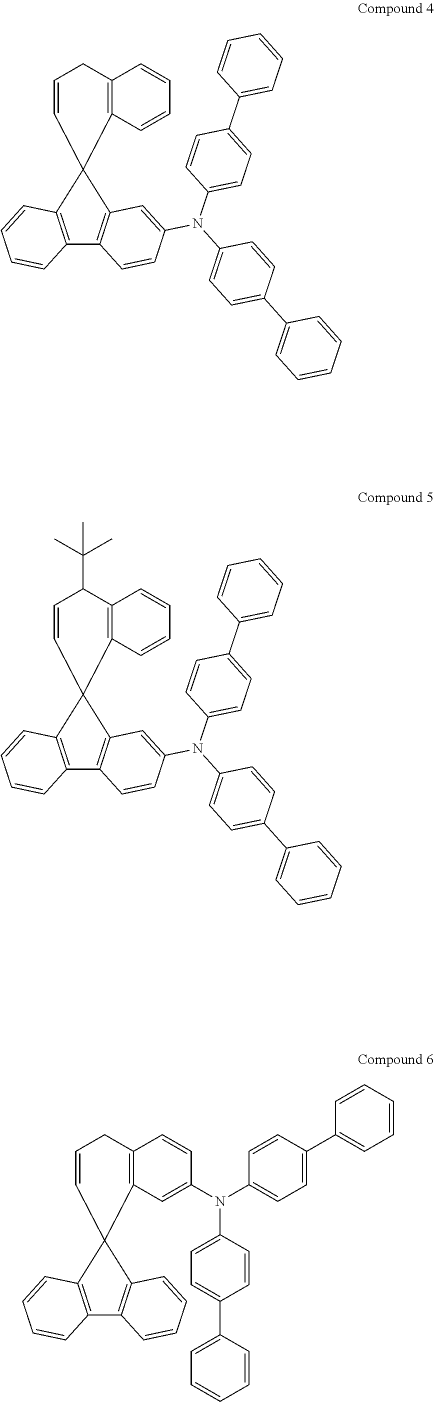 [SMILES: C1=CC2(c3ccccc3C1)c1ccccc1-c1ccc(N(c3ccc(-c4ccccc4)cc3)c3ccc(-c4ccccc4)cc3)cc12 ; CC(C)(C)C1C=CC2(c3ccccc3-c3ccc(N(c4ccc(-c5ccccc5)cc4)c4ccc(-c5ccccc5)cc4)cc32)c2ccccc21 ; C1=CC2(c3cc(N(c4ccc(-c5ccccc5)cc4)c4ccc(-c5ccccc5)cc4)ccc3C1)c1ccccc1-c1ccccc12]

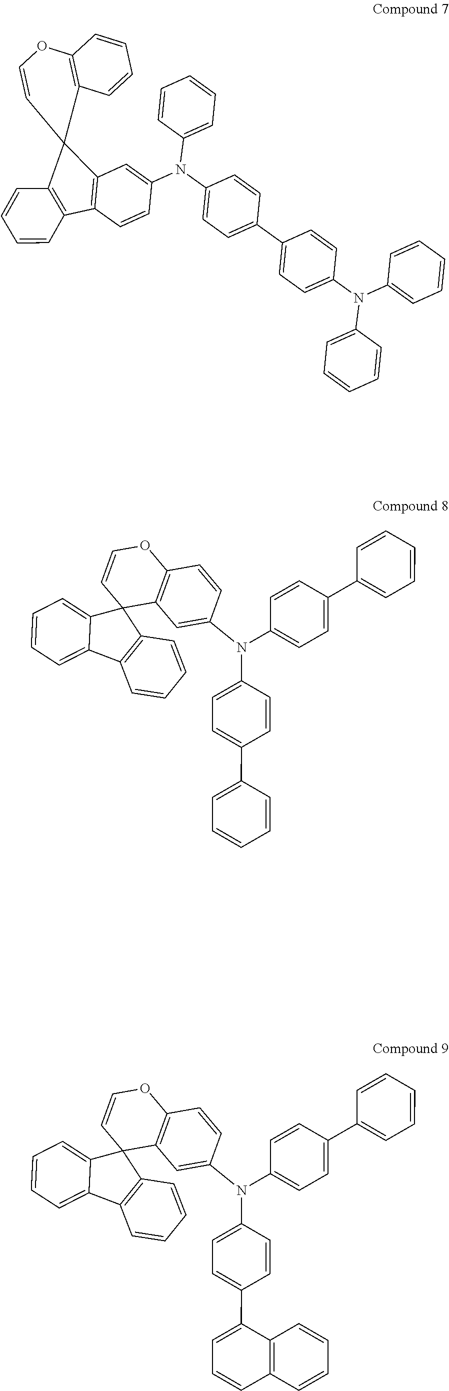 [SMILES: C1=CC2(c3ccccc3O1)c1ccccc1-c1ccc(N(c3ccccc3)c3ccc(-c4ccc(N(c5ccccc5)c5ccccc5)cc4)cc3)cc12 ; C1=CC2(c3cc(N(c4ccc(-c5ccccc5)cc4)c4ccc(-c5ccccc5)cc4)ccc3O1)c1ccccc1-c1ccccc12 ; C1=CC2(c3cc(N(c4ccc(-c5ccccc5)cc4)c4ccc(-c5cccc6ccccc56)cc4)ccc3O1)c1ccccc1-c1ccccc12]

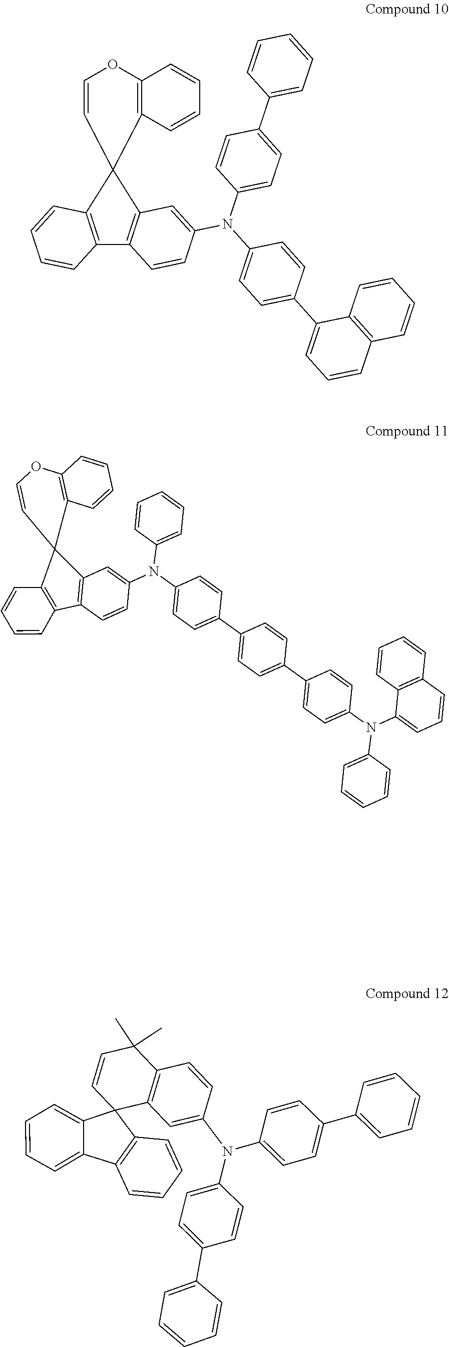 [SMILES: C1=CC2(c3ccccc3O1)c1ccccc1-c1ccc(N(c3ccc(-c4ccccc4)cc3)c3ccc(-c4cccc5ccccc45)cc3)cc12 ; C1=CC2(c3ccccc3O1)c1ccccc1-c1ccc(N(c3ccccc3)c3ccc(-c4ccc(-c5ccc(N(c6ccccc6)c6cccc7ccccc67)cc5)cc4)cc3)cc12 ; CC1(C)C=CC2(c3ccccc3-c3ccccc32)c2cc(N(c3ccc(-c4ccccc4)cc3)c3ccc(-c4ccccc4)cc3)ccc21]

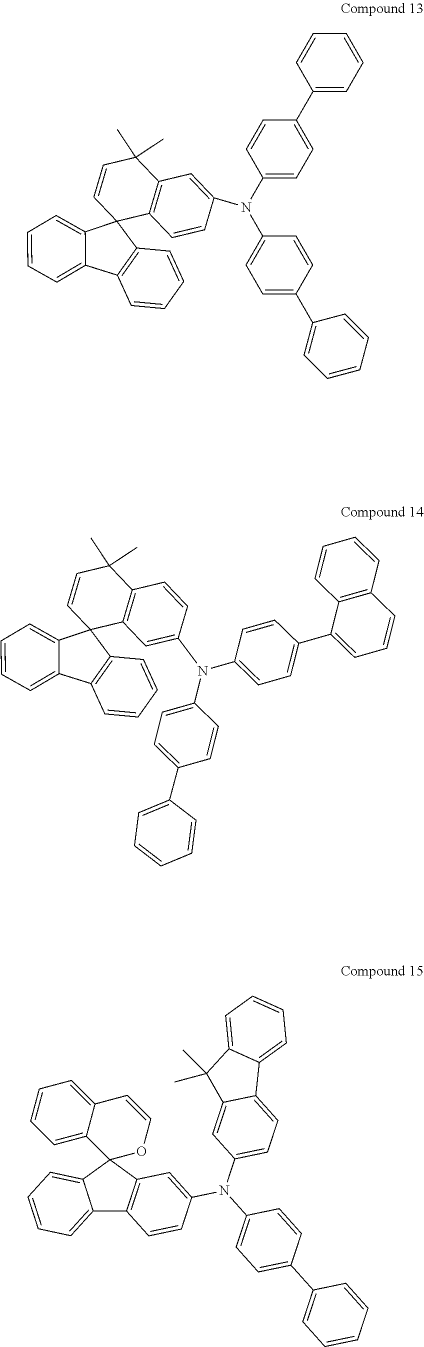 [SMILES: CC1(C)C=CC2(c3ccccc3-c3ccccc32)c2ccc(N(c3ccc(-c4ccccc4)cc3)c3ccc(-c4ccccc4)cc3)cc21 ; CC1(C)C=CC2(c3ccccc3-c3ccccc32)c2cc(N(c3ccc(-c4ccccc4)cc3)c3ccc(-c4cccc5ccccc45)cc3)ccc21 ; CC1(C)c2ccccc2-c2ccc(N(c3ccc(-c4ccccc4)cc3)c3ccc4c(c3)C3(OC=Cc5ccccc53)c3ccccc3-4)cc21]

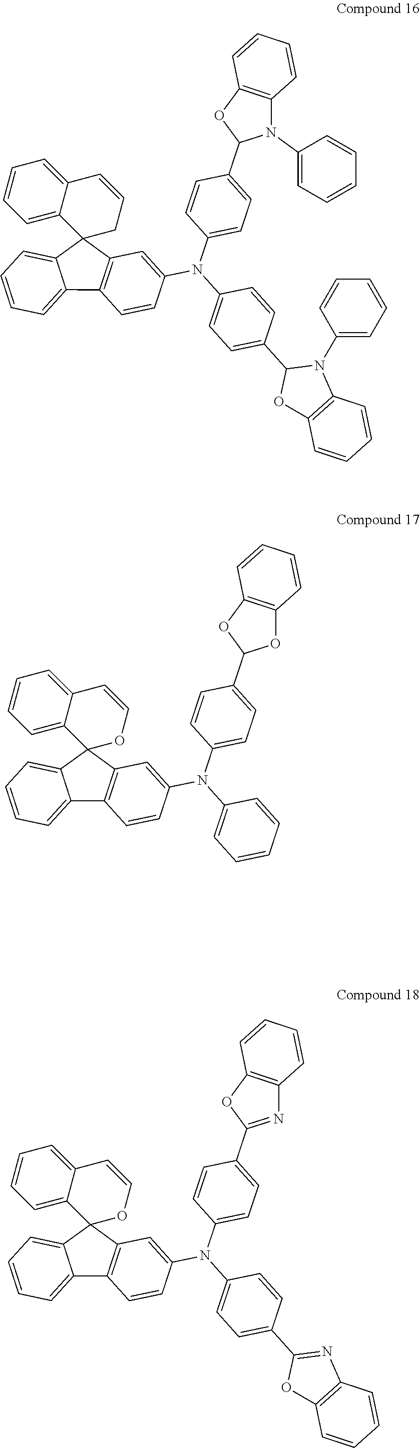 [SMILES: C1=Cc2ccccc2C2(C1)c1ccccc1-c1ccc(N(c3ccc(C4Oc5ccccc5N4c4ccccc4)cc3)c3ccc(C4Oc5ccccc5N4c4ccccc4)cc3)cc12 ; C1=Cc2ccccc2C2(O1)c1ccccc1-c1ccc(N(c3ccccc3)c3ccc(C4Oc5ccccc5O4)cc3)cc12 ; C1=Cc2ccccc2C2(O1)c1ccccc1-c1ccc(N(c3ccc(-c4nc5ccccc5o4)cc3)c3ccc(-c4nc5ccccc5o4)cc3)cc12]

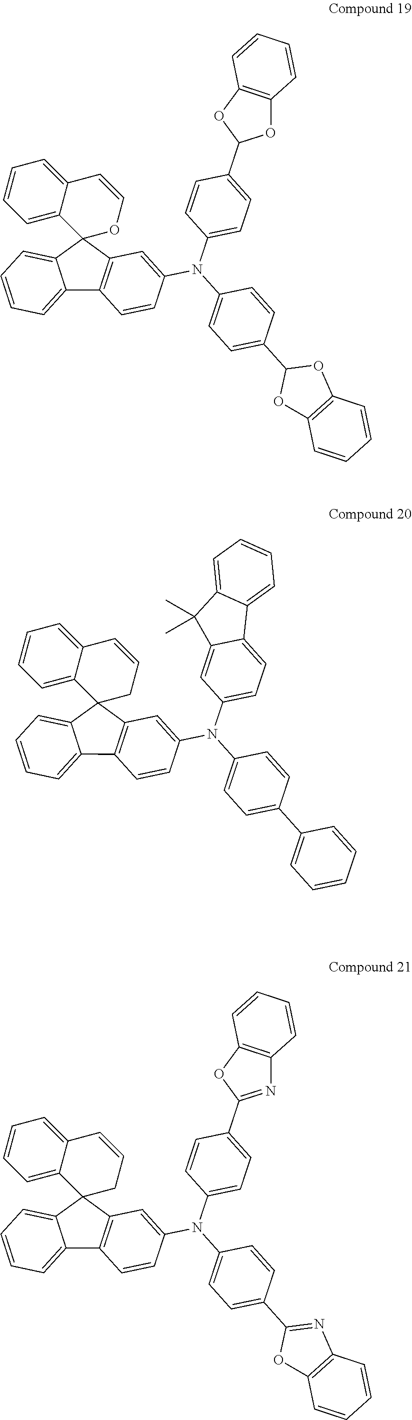 [SMILES: C1=Cc2ccccc2C2(O1)c1ccccc1-c1ccc(N(c3ccc(C4Oc5ccccc5O4)cc3)c3ccc(C4Oc5ccccc5O4)cc3)cc12 ; CC1(C)c2ccccc2-c2ccc(N(c3ccc(-c4ccccc4)cc3)c3ccc4c(c3)C3(CC=Cc5ccccc53)c3ccccc3-4)cc21 ; C1=Cc2ccccc2C2(C1)c1ccccc1-c1ccc(N(c3ccc(-c4nc5ccccc5o4)cc3)c3ccc(-c4nc5ccccc5o4)cc3)cc12]

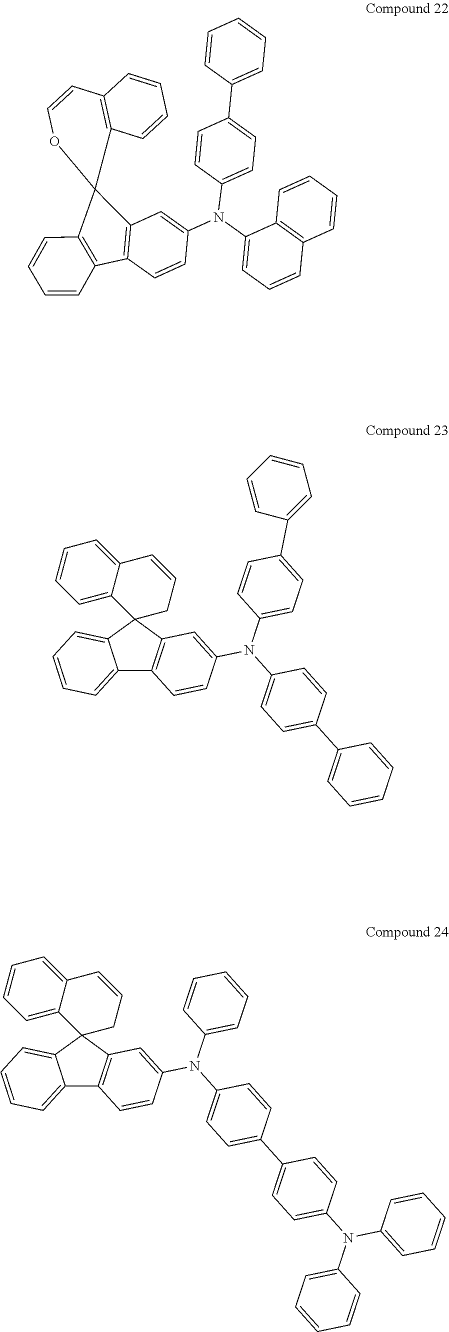 [SMILES: C1=Cc2ccccc2C2(O1)c1ccccc1-c1ccc(N(c3ccc(-c4ccccc4)cc3)c3cccc4ccccc34)cc12 ; C1=Cc2ccccc2C2(C1)c1ccccc1-c1ccc(N(c3ccc(-c4ccccc4)cc3)c3ccc(-c4ccccc4)cc3)cc12 ; C1=Cc2ccccc2C2(C1)c1ccccc1-c1ccc(N(c3ccccc3)c3ccc(-c4ccc(N(c5ccccc5)c5ccccc5)cc4)cc3)cc12]

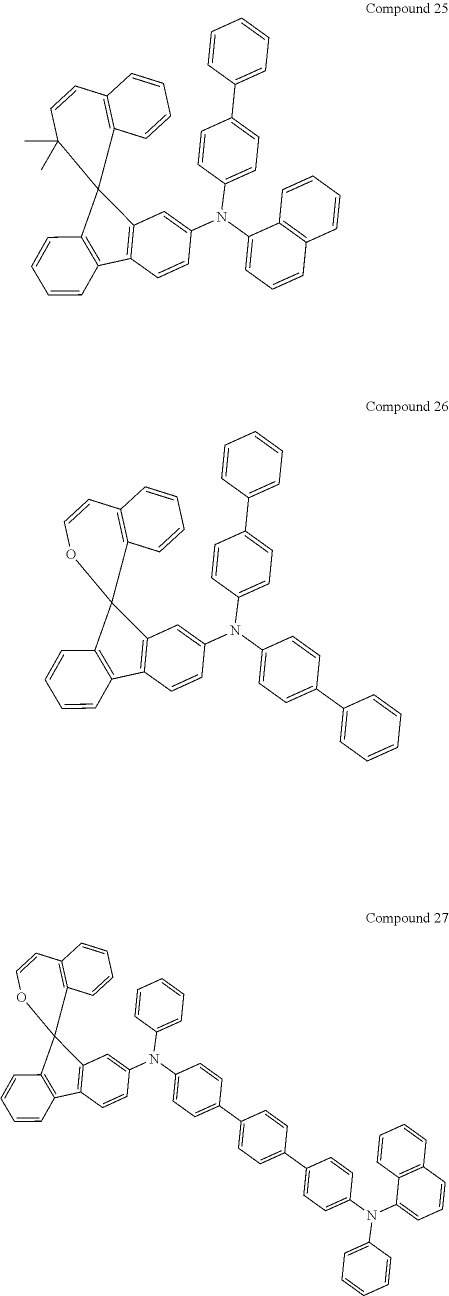 [SMILES: CC1(C)C=Cc2ccccc2C12c1ccccc1-c1ccc(N(c3ccc(-c4ccccc4)cc3)c3cccc4ccccc34)cc12 ; C1=Cc2ccccc2C2(O1)c1ccccc1-c1ccc(N(c3ccc(-c4ccccc4)cc3)c3ccc(-c4ccccc4)cc3)cc12 ; C1=Cc2ccccc2C2(O1)c1ccccc1-c1ccc(N(c3ccccc3)c3ccc(-c4ccc(-c5ccc(N(c6ccccc6)c6cccc7ccccc67)cc5)cc4)cc3)cc12]

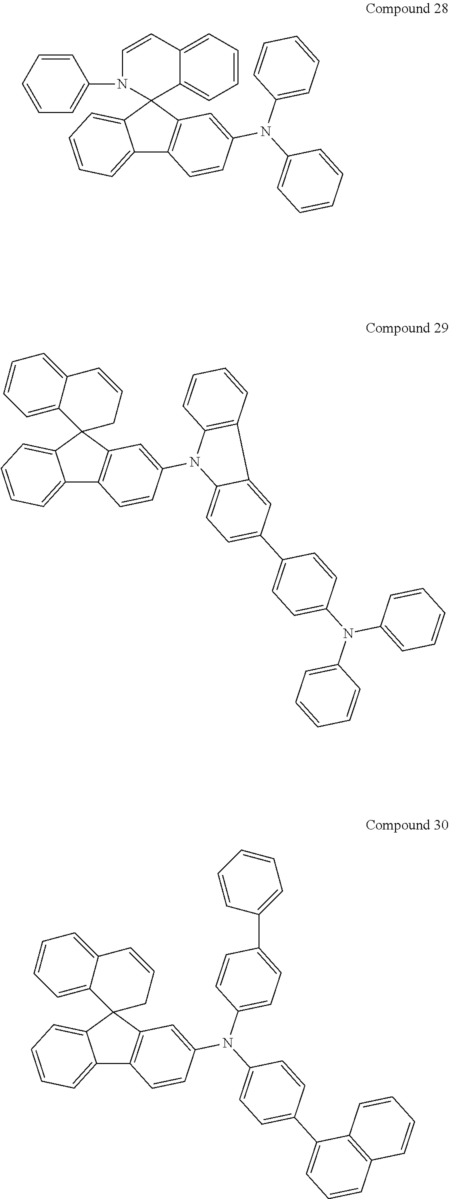 [SMILES: C1=CN(c2ccccc2)C2(c3ccccc31)c1ccccc1-c1ccc(N(c3ccccc3)c3ccccc3)cc12 ; C1=Cc2ccccc2C2(C1)c1ccccc1-c1ccc(-n3c4ccccc4c4cc(-c5ccc(N(c6ccccc6)c6ccccc6)cc5)ccc43)cc12 ; C1=Cc2ccccc2C2(C1)c1ccccc1-c1ccc(N(c3ccc(-c4ccccc4)cc3)c3ccc(-c4cccc5ccccc45)cc3)cc12]

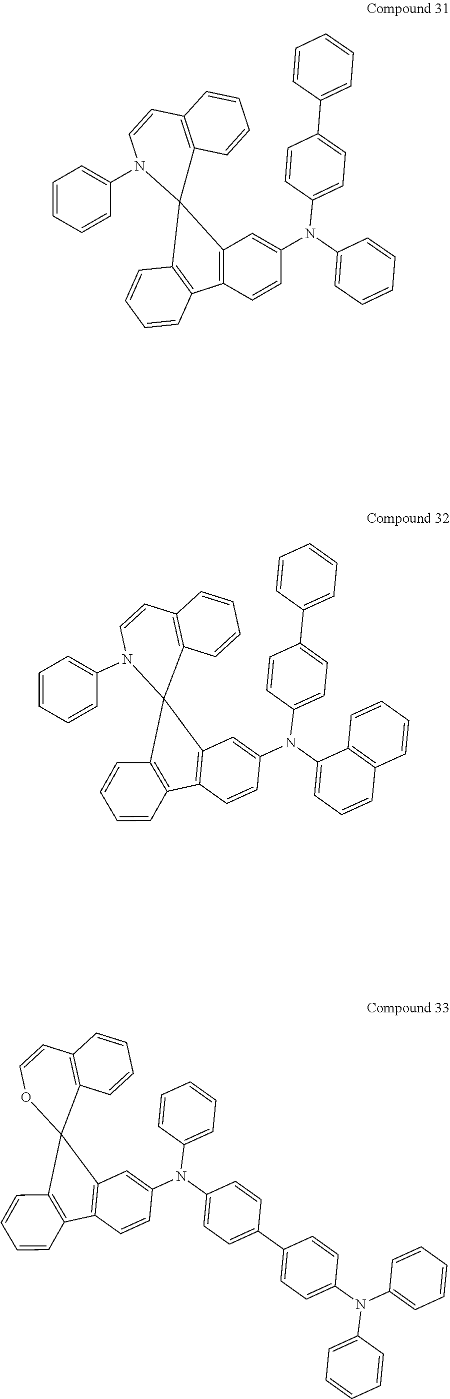 [SMILES: C1=CN(c2ccccc2)C2(c3ccccc31)c1ccccc1-c1ccc(N(c3ccccc3)c3ccc(-c4ccccc4)cc3)cc12 ; C1=CN(c2ccccc2)C2(c3ccccc31)c1ccccc1-c1ccc(N(c3ccc(-c4ccccc4)cc3)c3cccc4ccccc34)cc12 ; C1=Cc2ccccc2C2(O1)c1ccccc1-c1ccc(N(c3ccccc3)c3ccc(-c4ccc(N(c5ccccc5)c5ccccc5)cc4)cc3)cc12]

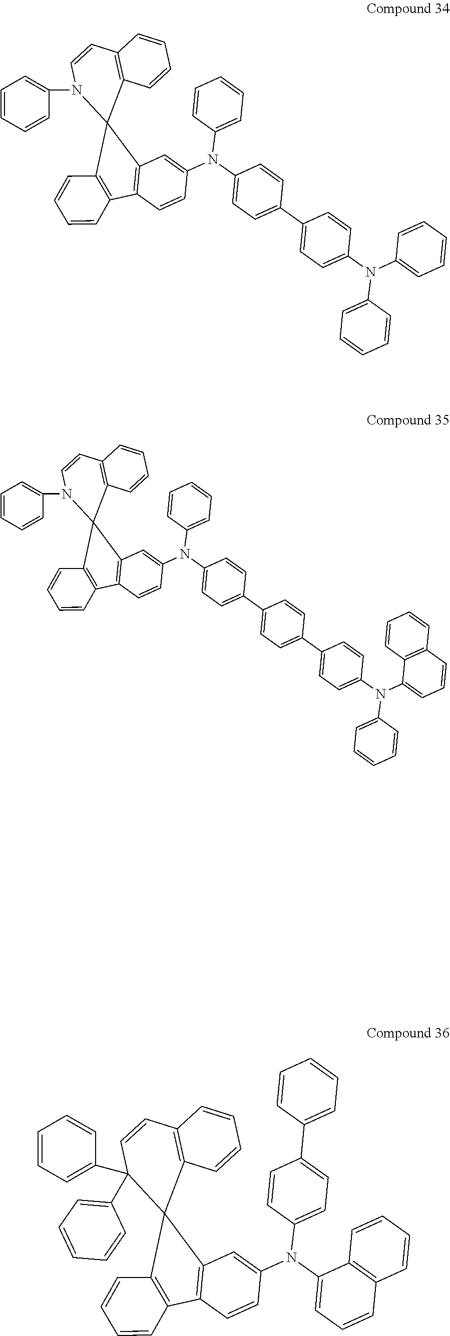 [SMILES: C1=CN(c2ccccc2)C2(c3ccccc31)c1ccccc1-c1ccc(N(c3ccccc3)c3ccc(-c4ccc(N(c5ccccc5)c5ccccc5)cc4)cc3)cc12 ; C1=CN(c2ccccc2)C2(c3ccccc31)c1ccccc1-c1ccc(N(c3ccccc3)c3ccc(-c4ccc(-c5ccc(N(c6ccccc6)c6cccc7ccccc67)cc5)cc4)cc3)cc12 ; C1=CC(c2ccccc2)(c2ccccc2)C2(c3ccccc31)c1ccccc1-c1ccc(N(c3ccc(-c4ccccc4)cc3)c3cccc4ccccc34)cc12]

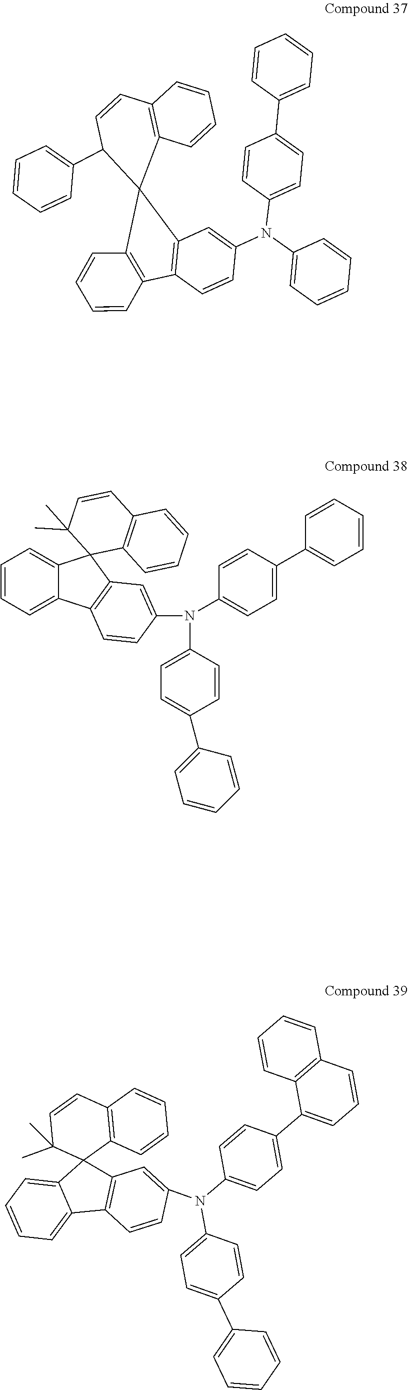 [SMILES: C1=CC(c2ccccc2)C2(c3ccccc31)c1ccccc1-c1ccc(N(c3ccccc3)c3ccc(-c4ccccc4)cc3)cc12 ; CC1(C)C=Cc2ccccc2C12c1ccccc1-c1ccc(N(c3ccc(-c4ccccc4)cc3)c3ccc(-c4ccccc4)cc3)cc12 ; CC1(C)C=Cc2ccccc2C12c1ccccc1-c1ccc(N(c3ccc(-c4ccccc4)cc3)c3ccc(-c4cccc5ccccc45)cc3)cc12]

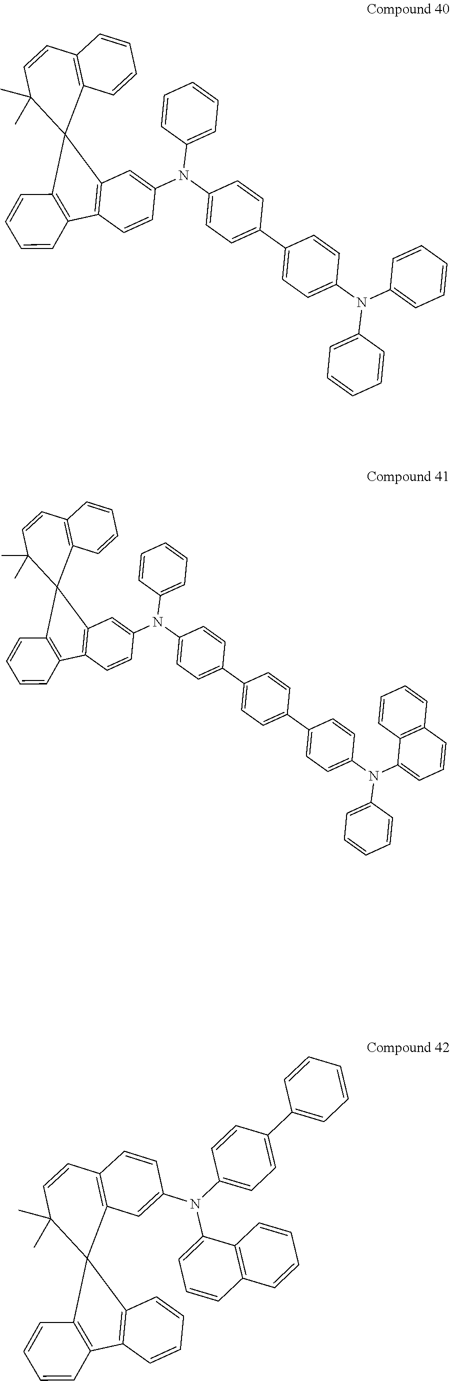 [SMILES: CC1(C)C=Cc2ccccc2C12c1ccccc1-c1ccc(N(c3ccccc3)c3ccc(-c4ccc(N(c5ccccc5)c5ccccc5)cc4)cc3)cc12 ; CC1(C)C=Cc2ccccc2C12c1ccccc1-c1ccc(N(c3ccccc3)c3ccc(-c4ccc(-c5ccc(N(c6ccccc6)c6cccc7ccccc67)cc5)cc4)cc3)cc12 ; CC1(C)C=Cc2ccc(N(c3ccc(-c4ccccc4)cc3)c3cccc4ccccc34)cc2C12c1ccccc1-c1ccccc12]

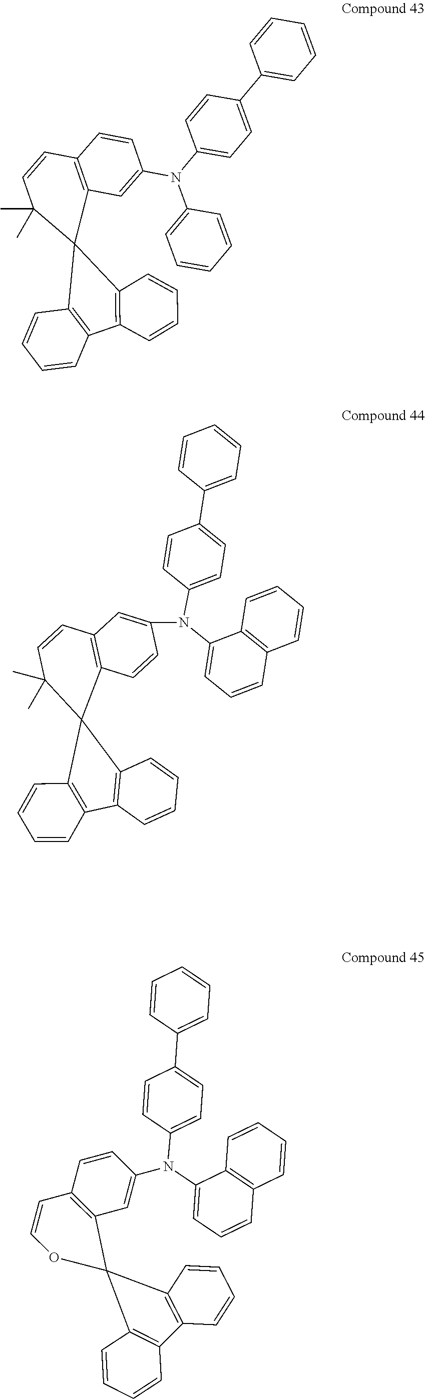 [SMILES: CC1(C)C=Cc2ccc(N(c3ccccc3)c3ccc(-c4ccccc4)cc3)cc2C12c1ccccc1-c1ccccc12 ; CC1(C)C=Cc2cc(N(c3ccc(-c4ccccc4)cc3)c3cccc4ccccc34)ccc2C12c1ccccc1-c1ccccc12 ; C1=Cc2ccc(N(c3ccc(-c4ccccc4)cc3)c3cccc4ccccc34)cc2C2(O1)c1ccccc1-c1ccccc12]

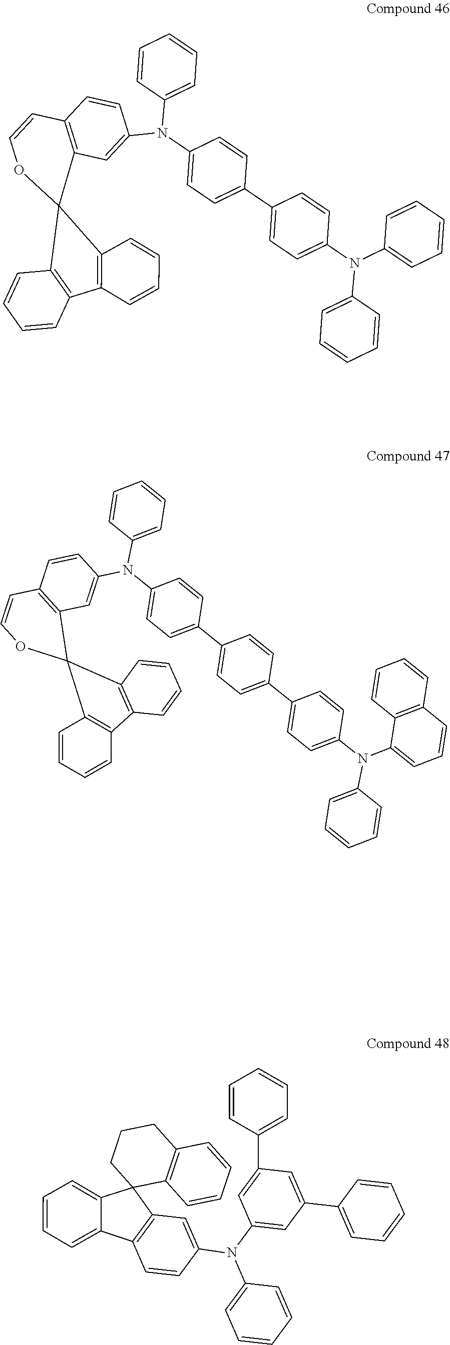 [SMILES: C1=Cc2ccc(N(c3ccccc3)c3ccc(-c4ccc(N(c5ccccc5)c5ccccc5)cc4)cc3)cc2C2(O1)c1ccccc1-c1ccccc12 ; C1=Cc2ccc(N(c3ccccc3)c3ccc(-c4ccc(-c5ccc(N(c6ccccc6)c6cccc7ccccc67)cc5)cc4)cc3)cc2C2(O1)c1ccccc1-c1ccccc12 ; c1ccc(-c2cc(-c3ccccc3)cc(N(c3ccccc3)c3ccc4c(c3)C3(CCCc5ccccc53)c3ccccc3-4)c2)cc1]

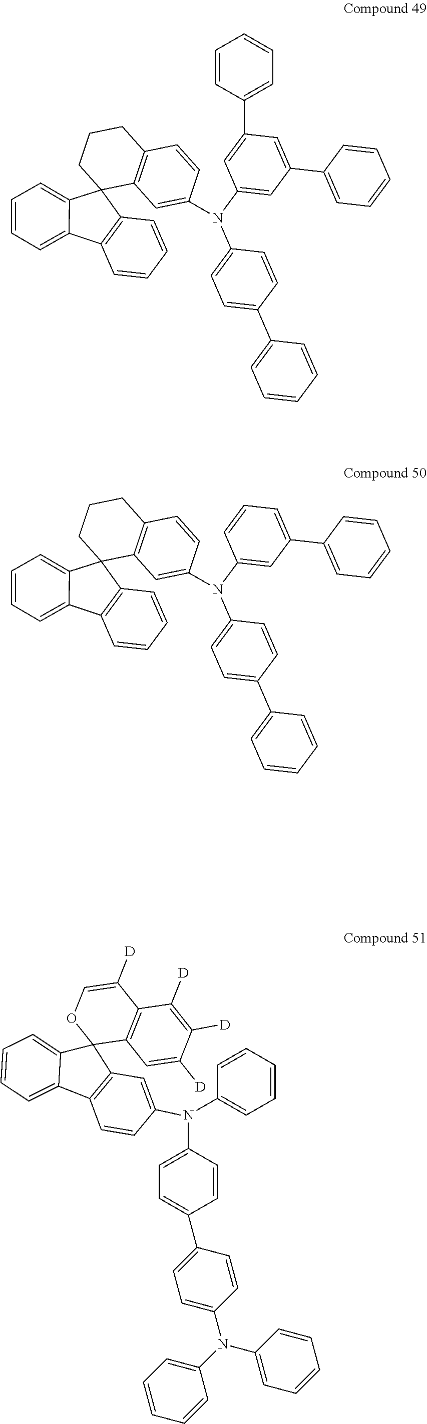 [SMILES: c1ccc(-c2ccc(N(c3cc(-c4ccccc4)cc(-c4ccccc4)c3)c3ccc4c(c3)C3(CCC4)c4ccccc4-c4ccccc43)cc2)cc1 ; c1ccc(-c2ccc(N(c3cccc(-c4ccccc4)c3)c3ccc4c(c3)C3(CCC4)c4ccccc4-c4ccccc43)cc2)cc1 ; [2H]C1=COC2(c3ccccc3-c3ccc(N(c4ccccc4)c4ccc(-c5ccc(N(c6ccccc6)c6ccccc6)cc5)cc4)cc32)c2cc([2H])c([2H])c([2H])c21]

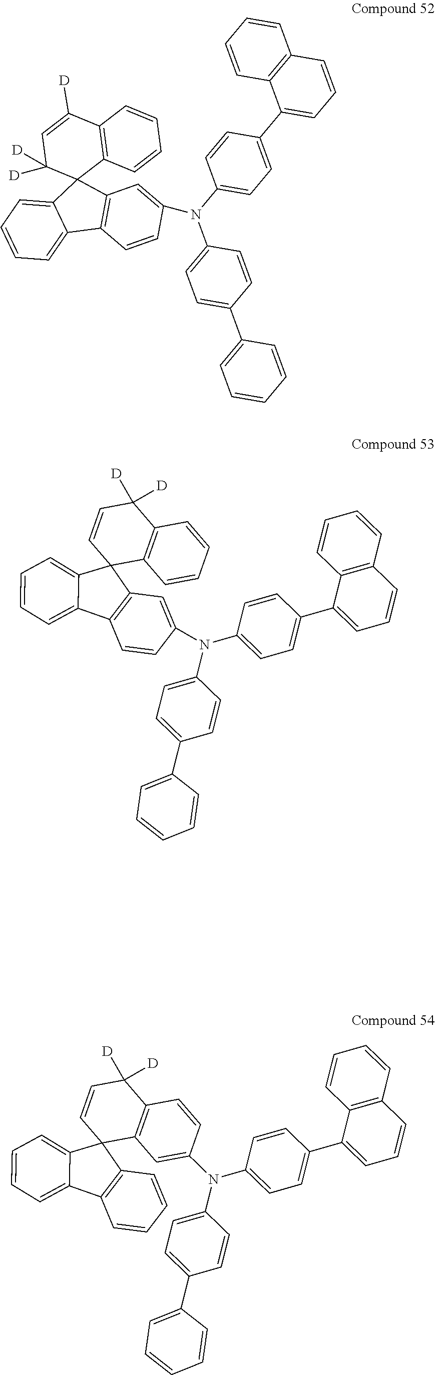 [SMILES: [2H]C1=CC([2H])([2H])C2(c3ccccc31)c1ccccc1-c1ccc(N(c3ccc(-c4ccccc4)cc3)c3ccc(-c4cccc5ccccc45)cc3)cc12 ; [2H]C1([2H])C=CC2(c3ccccc3-c3ccc(N(c4ccc(-c5ccccc5)cc4)c4ccc(-c5cccc6ccccc56)cc4)cc32)c2ccccc21 ; [2H]C1([2H])C=CC2(c3ccccc3-c3ccccc32)c2cc(N(c3ccc(-c4ccccc4)cc3)c3ccc(-c4cccc5ccccc45)cc3)ccc21]

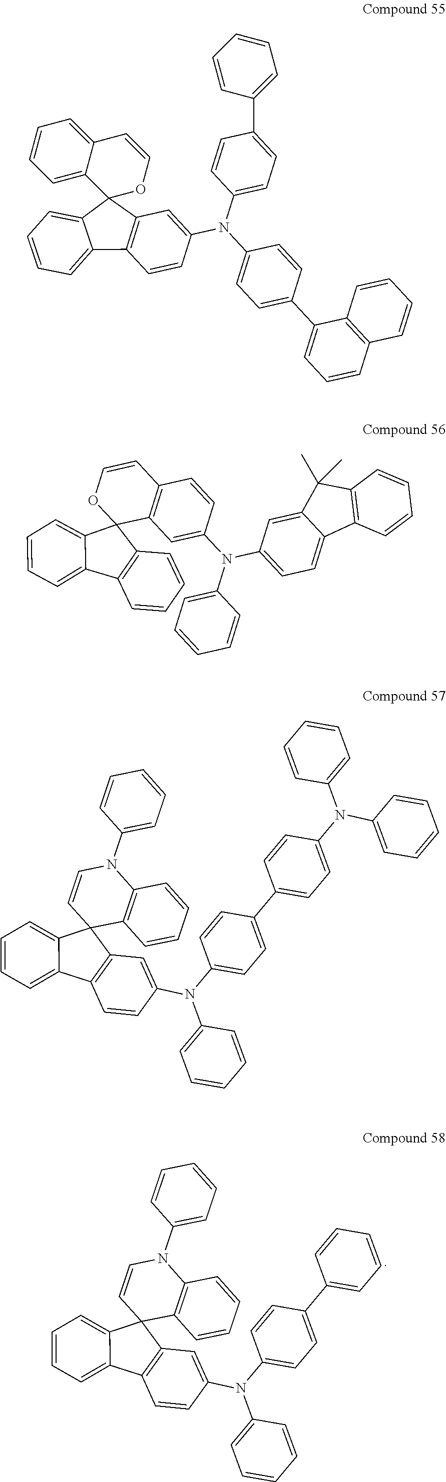 [SMILES: C1=Cc2ccccc2C2(O1)c1ccccc1-c1ccc(N(c3ccc(-c4ccccc4)cc3)c3ccc(-c4cccc5ccccc45)cc3)cc12 ; CC1(C)c2ccccc2-c2ccc(N(c3ccccc3)c3ccc4c(c3)C3(OC=C4)c4ccccc4-c4ccccc43)cc21 ; C1=CC2(c3ccccc3-c3ccc(N(c4ccccc4)c4ccc(-c5ccc(N(c6ccccc6)c6ccccc6)cc5)cc4)cc32)c2ccccc2N1c1ccccc1 ; C1=CC2(c3ccccc3-c3ccc(N(c4ccccc4)c4ccc(-c5ccccc5)cc4)cc32)c2ccccc2N1c1ccccc1]

16. The arylamine compound according to claim 1, having a glass transition temperature of 120° C. to 180° C., or 125° C. to 140° C., a highest occupied molecular orbital energy level of −4.6 eV to −5.7 eV, −4.6 eV to −4.9 eV, or −5.3 eV to −5.5 eV, and a triplet energy level of 1.8 eV to 2.6 eV, 2.1 eV to 2.6 eV, or 2.4 eV to 2.6 eV.

17-22. (canceled)

23. Use of the diarylamine compound according to claim 1 as a hole injection material, a hole transport material, or an electron block material.

24-25. (canceled)

26. An electroluminescent device, comprising the arylamine compound according to claim 1.

27. The electroluminescent device of claim 26, comprising a hole injection layer, a hole transport layer, and an electron block layer, wherein,
the hole injection layer and the hole transport layer each comprise the arylamine compound; or,
the hole injection layer and the electron block layer each comprise the arylamine compound; or,
the hole injection layer, the hole transport layer, and the electron block layer each comprise the arylamine compound.

28. A display apparatus, comprising the electroluminescent device according to claim 26.
